SEQUENCE LISTING (17 sequences)

SEQ ID NO: 1 - DNA, 1768 bp, Enterococcus faecium
gatatcgttacgcttcatgtgccgctcaatacggatacgcactatattatcagccacgaacaaatacagagaatgaagcaaggagcatttcttatcaatactgggcgcggtccacttgtagatacctatgagttggttaaagcattagaaaacgggaaactgggcggtgccgcattggatgtattggaaggagaggaagagtttttctactctgattgcacccaaaaaccaattgataatcaatttttacttaaacttcaaagaatgcctaacgtgataatcacaccgcatacggcctattataccgagcaagcgttgcgtgataccgttgaaaaaaccattaaaaactgtttggattttgaaaggagacaggagcatgaatagaataaaagttgcaatactgtttgggggttgctcagaggagcatgacgtatcggtaaaatctgcaatagagatagccgctaacattaataaagaaaaatacgagccgttatacattggaattacgaaatctggtgtatggaaaatgtgcgaaaaaccttgcgcggaatgggaaaacgacaattgctattcagctgtactctcgccggataaaaaaatgcacggattacttgttaaaaagaaccatgaatatgaaatcaaccatgttgatgtagcattttcagctttgcatggcaagtcaggtgaagatggatccatacaaggtctgtttgaattgtccggtatcccttttgtaggctgcgatattcaaagctcagcaatttgtatggacaaatcgttgacatacatcgttgcgaaaaatgctgggatagctactcccgccttttgggttattaataaagatgataggccggtggcagctacgtttacctatcctgtttttgttaagccggcgcgttcaggctcatccttcggtgtgaaaaaagtcaatagcgcggacgaattggactacgcaattgaatcggcaagacaatatgacagcaaaatcttaattgagcaggctgtttcgggctgtgaggtcggttgtgcggtattgggaaacagtgccgcgttagttgttggcgaggtggaccaaatcaggctgcagtacggaatctttcgtattcatcaggaagtcgagccggaaaaaggctctgaaaacgcagttataaccgttcccgcagacctttcagcagaggagcgaggacggatacaggaaacggcaaaaaaaatatataaagcgctcggctgtagaggtctagcccgtgtggatatgtttttacaagataacggccgcattgtactgaacgaagtcaatactctgcccggtttcacgtcatacagtcgttatccccgtatgatggccgctgcaggtattgcacttcccgaactgattgaccgcttgatcgtattagcgttaaaggggtgataagcatggaaataggatttacttttttagatgaaatagtacacggtgttcgttgggacgctaaatatgccacttgggataatttcaccggaaaaccggttgacggttatgaagtaaatcgcattgtagggacatacgagttggctgaatcgcttttgaaggcaaaagaactggctgctacccaagggtacggattgcttctatgggacggttaccgtcctaagcgtgctgtaaactgttttatgcaatgggctgcacagccggaaaataacctgacaaaggaaagttattatcccaatattgaccgaactgagatgatttcaaaaggatacgtggcttcaaaatcaagccatagccgcg

SEQ ID NO: 2 - DNA, 18 bp, Enterococcus faecium
ccggtggcagctacgttt

SEQ ID NO: 3 - DNA, 27 bp, Enterococcus faecium
cctatcctgtttttgttaagccggcgc

SEQ ID NO: 4 - DNA, 20 bp, Enterococcus faecium
caccgaaggatgagcctgaa

SEQ ID NO: 5 - DNA, 1090 bp, Enterococcus faecalis
tgctgcgagataccacagaaaacaatcaggaattgtcttaactttgaaaggagtttacagcatgaataaaataaaagtcgcaattatcttcggcggttgctcggaggaacatgatgtgtcggtaaaatccgcaatagaaattgctgcgaacattaatactgaaaaattcgatccgcactacatcggaattacaaaaaacggcgtatggaagctatgcaagaagccatgtacggaatgggaagccgatagtctccccgccatattctccccggataggaaaacgcatggtctgcttgtcatgaaagaaagagaatacgaaactcggcgtattgacgtggctttcccggttttgcatggcaaatgcggggaggatggtgcgatacagggtctgtttgaattgtctggtatcccctatgtaggctgcgatattcaaagctccgcagcttgcatggacaaatcactggcctacattcttacaaaaaatgcgggcatcgccgtccccgaatttcaaatgattgaaaaaggtgacaaaccggaggcgaggacgcttacctaccctgtctttgtgaagccggcacggtcaggttcgtcctttggcgtaaccaaagtaaacagtacggaagaactaaacgctgcgatagaagcagcaggacaatatgatggaaaaatcttaattgagcaagcgatttcgggctgtgaggtcggctgcgcggtcatgggaaacgaggatgatttgattgtcggcgaagtggatcaaatccggttgagccacggtatcttccgcatccatcaggaaaacgagccggaaaaaggctcagagaatgcgatgattatcgttccagcagacattccggtcgaggaacgaaatcgggtgcaagaaacggcaaagaaagtatatcgggtgcttggatgcagagggcttgctcgtgttgatctttttttgcaggaggatggcggcatcgttctaaacgaggtcaataccctgcccggttttacatcgtacagccgctatccacgcatggcggctgccgcaggaatcacgcttcccgcactaattgacagcctgattacattggcgatagagaggtga

SEQ ID NO: 6 - DNA, 18 bp, Enterococcus faecalis
cgacctcacagcccgaaa

SEQ ID NO: 7 - DNA, 18 bp, Enterococcus faecalis
cgcttgctcaattaagat

SEQ ID NO: 8 - DNA, 21 bp, Enterococcus faecalis
cggcaggacaatatgatggaa

SEQ ID NO: 9 - DNA, 21 bp, Enterococcus faecalis
cagcaggacaatatgatggaa

SEQ ID NO: 10 - DNA, 801 bp, Enterococcus faecium
aacgatgccgccatcctcctgcaaaaaaagatcaacacgggcaagccctctgcatccaagcacccgatatactttctttgccgtttcctgcacccgatttcgttcctcgaccggaatgtctgcgggaactgtaatcatcgcattttctgagcctttttccggctcgttttcctgatggatgcggaagataccgtggctcagccggatttgatccacttcgccgacaatcaaatcatcctcgttccccatgaccgcacacccgacctcacagcccgaaatcgcttgctcaattaagatttttccatcatattgtcctgccgcttctatcgcagcgttaagttcttccgtaccgtttactttggttacgccaaaggacgaacctgaccgtgccggcttcacaaagacagggtaggtaagcgcacccgcctccggcttgtcacctttatcaatcatttgaaattcgggaacggcgatgcccgcattttttgtaagaatgtaggccagtgatttgtccatgcaagctgcggagctttgaatatcacagcccacataggggataccagacaatacaaacagcccctgtatcgcaccatcctccccgcatttgccatgcaaaaccgggaaagccacatcaatacgccgtgtttcgtattcgctttctttcatgacaagcagcccatgcgttttcctatccggggagagtatggcggggagactgtcggcttcccattccgtacatggcttcttgcatagcttccatacaccgttttttgtaattccgatgtagtgcggatcgaattt

SEQ ID NO: 11 - DNA, 801 bp, Enterococcus faecium
aacgatgccgccatcctcctgcaaaaaaagatcaacacgagcaagccctctgcatccaagcacccgatatactttctttgccgtttcttgcacccgatttcgttcctcgaccggaatgtctgctggaacgataatcatcgcattctctgagcctttttccggctcgttttcctgatggatgcggaagataccgtggctcaaccggatttgatccacttcgccgacaatcaaatcatcctcgtttcccatgaccgcgcagccgacctcacagcccgaaatcgcttgctcaattaagatttttccatcatattgtcctgctgcttctatcgcagcgtttagttcttccgtactgtttactttggttacgccaaaggacgaacctgaccgtgccggcttcacaaagacagggtaggtaagcgtcctcgcctccggtttgtcacctttttcaatcatttgaaattcggggacggcgatgcccgcattttttgtaagaatgtaggccagtgatttgtccatgcaagctgcggagctttgaatatcgcagcctacataggggataccagacaattcaaacagaccctgtatcgcaccatcctccccgcatttgccatgcaaaaccgggaaagccacgtcaatacgccgagtttcgtattctctttctttcatgacaagcagaccatgcgttttcctatccggggagaatatggcggggagactatcggcttcccattccgtacatggcttcttgcatagcttccatacgccgttttttgtaattccgatgtagtgcggatcgaattt

SEQ ID NO: 12 - DNA, 801 bp, Enterococcus faecium
aacgatgccgccatcctcctgcaaaaaaagatcaacacgggcaagccctctgcatccaagcacccgatatactttctttgccgtttcctgcacccgatttcgttcctcgaccggaatgtctgcgggaactgtaatcatcgcattttctgagcctttttccggctcgttttcctgatggatgcggaagataccgtggctcagccggatttgatccacttcgccgacaatcaaatcatcctcgttccccatgaccgcacacccgacctcacagcccgaaatcgcttgctcaattaagatttttccatcatattgtcctgccgcttctatcgcagcgttaagttcttccgtaccgtttactttggttacgccaaaggacgaacctgaccgtgccggcttcacaaagacagggtaggtaagcgcacccgcctccggcttgtcacctttatcaataatttgaaattcgggaacggcgatgcccgcattttttgtaagaatgtaggccagtgatttgtccatgcaagctgcggagctttgaatatcacagcccacataggggataccagacaatacaaacagcccctgtatcgcaccatcctccccgcatttgccatgcaaaaccgggaaagccacatcaatacgccgtgtttcgtattcgctttctttcatgacaagcagcccatgcgttttcctatccggggagagtatggcggggagactgtcggcttcccattccgtacatggcttcttgcatagcttccatacaccgttttttgtaattccgatgtagtgcggatcgaattt

SEQ ID NO: 13 - DNA, 801 bp, Enterococcus faecium; identical to SEQ ID NO: 12

SEQ ID NO: 14 - DNA, 801 bp, Enterococcus faecium; identical to SEQ ID NO: 12

SEQ ID NO: 15 - DNA, 630 bp, Enterococcus faecalis
gaggatgggtgcatccagggactgtttgaattgtctggtatcccctatgtgggctgtgatattcaaagctccgcagcttgcatggacaaatcactggcctacattcttacaaaaaatgcgggcatcgccgttcccgaatttcaaatgattgataaaggtgacaagccggaggcgggtgcgcttacctaccctgtctttgtgaagccggcacggtcaggttcgtcctttggcgtaaccaaagtaaacggtacggaagaacttaacgctgcgatagaagcggcaggacaatatgatggaaaaatcttaattgagcaagcgatttcgggctgtgaggtcgggtgtgcggtcatggggaacgaggatgatttgattgtcggcgaagtggatcaaatccggctgagccacggtatcttccgcatccatcaggaaaacgagccggaaaaaggctcagaaaatgcgatgattacagttcccgcagacattccggtcgaggaacgaaatcgggtgcaggaaacggcaaagaaagtatatcgggtgcttggatgcagagggcttgcccgtgttgatctttttttgcaggaggatggcggcatcgttctaaatgaggtcaacaccctgcccggcttcacg

SEQ ID NO: 16 - DNA, 783 bp, Enterococcus faecalis
gaaaaattcgatccgcactacatcggaattacaaaaaggggtgtatggaagctatgcaagaagccatgtacggaatgggaagccgacagtctccccgccatactctccccggataggaaaacgcatggtctgcttgtcatgaaagaaagcgaatacgaaacacggcgtattgatgtggctttcccagttttgcatggcaaatgcggggaggacggtgcgatacagggtttatttgaattgtctggcatcccctatgtgggctgcgatattcaaagctccgcagcttgcatggacaaatcactggcctacattcttacaaaaaatgcgggcatcgccgttcccgaatttcaaatgattgataaaggtgacaagccggagacgggtgcgcttacctaccctgtctttgtgaagccggcacggtcaggttcgtcctttggcttaaccaaagtaaacggtacggaagaacttaacgctgcgatagaagcggcaggacaatatgatggaaaaatcttaattgagcaagcgatttcgggctgtgaggtcggctgtgcggttatggggaacgaggatgatttgattgtcggcgaagtggatcaaatccggctgagccatggtatcttccgcatccatcaggaaaacgagccggaaaaaggatcagagaatgcgatgattaccgttcctgcagacatcccagtcggggaacgaaatcgggtgcaggaaacggcaaagaaagtatatcgggcgcttggatgcagagggcttgcccgtgttgatctttttttg

SEQ ID NO: 17 - DNA, 801 bp, Artificial Sequence
  A synthetic consensus sequence
aacgatgccgccatcctcctgcaaaaaaagatcaacacgggcaagccctctgcatccaagcacccgatatactttctttgccgtttcctgcacccgatttcgttcctcgaccggaatgtctgcgggaactgtaatcatcgcattttctgagcctttttccggctcgttttcctgatggatgcggaagataccgtggctcagccggatttgatccacttcgccgacaatcaaatcatcctcgttccccatgaccgcacacccgacctcacagcccgaaatcgcttgctcaattaagatttttccatcatattgtcctgccgcttctatcgcagcgttaagttcttccgtaccgtttactttggttacgccaaaggacgaacctgaccgtgccggcttcacaaagacagggtaggtaagcgcacccgcctccggcttgtcacctttatcaatcatttgaaattcgggaacggcgatgcccgcattttttgtaagaatgtaggccagtgatttgtccatgcaagctgcggagctttgaatatcacagcccacataggggataccagacaattcaaacagcccctgtatcgcaccatcctccccgcatttgccatgcaaaaccgggaaagccacatcaatacgccgtgtttcgtattcgctttctttcatgacaagcagcccatgcgttttcctatccggggagagtatggcggggagactgtcggcttcccattccgtacatggcttcttgcatagcttccatacaccgttttttgtaattccgatgtagtgcggatcgaattt

(19) United States
(12) Patent Application Publication
(10) Pub. No.: US 2005/0058985 A1
(43) Pub. Date: Mar. 17, 2005

(54) Method and kit for identifying vancomycin-resistant enterococcus

(76) Inventor: Kirsty Jane Dodgson, Iowa City, IA (US)

Correspondence Address: Schwegman, Lundberg, Woessner & Kluth, P.A., P.O. Box 2938, Minneapolis, MN, 55402, US

(21) Appl. No.: 10/661,094

(22) Filed: Sep. 12, 2003

(51) Int. Cl. C12Q 1/70; C12Q 1/68

(52) U.S. Cl. 435/5; 435/6

(57) ABSTRACT

The invention provides a method to process samples for DNA detection and a method to identify the vancomycin resistance gene status of an organism.

### BACKGROUND OF THE INVENTION

Three pressures face the routine microbiology laboratory: increasing specimen numbers, principally for infection control purposes; a need to report prompt results; and shrinking budgets. Standard culture screening protocols typically require 3-5 days to issue final results (Sahm et al., 1997; Van Horn et al., 1996) because bacterial identification requires conventional biochemical tests. Other types of assays have been developed into more rapid diagnostic tools, e.g., immunoassays, including radioimmunoassays, enzyme-linked immunoassays, and latex agglutination and immunoblotting assays. Moreover, polynucleotide-based assays are rapidly gaining popularity in clinical laboratory practice.

For example, nucleic acid hybridization assays have been developed to detect microorganisms, and more recent advances in signal and target amplification have introduced the era of molecular diagnostics based on the use of oligonucleotide probes. Generally, a probe is a single-stranded polynucleotide having some degree of complementarity with a nucleic acid sequence that is to be detected (“target sequence”). A double-stranded nucleic acid hybrid between the probe and the target sequence results if the target sequence is contacted under hybridization-promoting conditions with a probe having a sufficient number of contiguous bases complementary to the target sequence. DNA/DNA, RNA/DNA or RNA/RNA hybrids may thus be formed under appropriate conditions. Probes commonly are labeled with a detectable moiety such as a radioisotope, a ligand, or a calorimetric, fluorometric or chemiluminescent moiety to facilitate the detection of hybrids.

Enterococci have been recognized as an important cause of nosocomial infection for the past two decades (Murray, 1990). High level resistance to vancomycin in enterococci is carried on a transposable gene cassette (Quintiliani et al., 1996; Evers et al., 1996; Arthur et al., 1992) with two distinct phenotypes; VanA (inducible resistance to both vancomycin and teicoplanin) and VanB (inducible resistance to vancomyin, but not teicoplanin). Studies linked higher morbidity and mortality rates to vancomycin resistance enterococci (VRE) infections and the number of isolates of enterococci resistant to vancomycin has increased dramatically in the last decade (Centers for Disease Control, 1993; Bemston et al., (1998). Because the organism can be transferred by nosocomial spread and remains viable in the environment, VRE containment protocols that include surveillance have been established (Centers for Disease Control, 1995). Nevertheless, these surveillance programs can be time-consuming, as culture requires 72-96 hours, as well as costly, to the clinical microbiology laboratory. Moreover, culture for VRE has a documented sensitivity of 58%, which is problematic.

A number of recent reports have focused on applying polymerase chain reaction (PCR) technology to detect VRE in a more timely manner. Some of these assays have been performed directly on clinical specimens (Petrich et al., 1999; Satake et al., 1997), while others have used an enrichment step in an overnight broth (Satake et al., 1997) or on selective media (Sahm et al., 1997). To minimize turnaround time, the ideal protocol would be PCR performed directly on the specimen. Nevertheless, one issue with direct amplification is that specimen preparation remains technically demanding and may not be suitable for the routine technologist (Petrich et al., 1999). A second issue is that a cultured specimen may still be required for identification at the species level and for epidemiologic typing. An enrichment culture of the VRE specimen would provide a simple specimen preparation, presumably fewer amplification inhibitors, and a cultured isolate available for subsequent confirmation.

One example of a polynucleotide-based assay for VRE is described in Petrich et al. (1999; 2000). In that assay, denatured biotinylated PCR amplicons are mixed with a fluorescein-labeled (FITC) detector probe and the mixture transferred to streptavidin-coated microtiter wells. After incubation and washing, a horseradish peroxidase (HRP)-conjugated antibody specific for FITC is added to detect VRE-specific amplicons.

However, there is a continuing need for rapid and accurate assays to detect VRE in patient samples.

### SUMMARY OF THE INVENTION

The present invention relates to polynucleotide-based methods, compositions, kits and devices that can be used to detect the vanA gene or the vanB gene, which genes are each associated with vancomycin resistance of microorganisms. As described hereinbelow, a rapid real time PCR for the detection of both vanA and vanB positive enterococci was developed. In particular, primers and fluorescent probes were designed that were specific for the vanA gene and all known vanB genes. Peri-rectal swabs for routine surveillance were cultured, then the swabs resuspended in 1 mL of phosphate buffer saline (PBS). 305 swabs were tested in total. An extract prepared from the PBS samples then was tested in a real time assay for both genes, and the PCR data compared to the current gold standard of culture. Using ATCC strains, the primers were shown to demonstrate specificity for each gene type. Moreover, the limit of detection was determined to be 80 cfu/mL for the vanA gene and 8 cfu/mL for the vanB gene. The PCR based assay detected 12 positive specimens that were not identified by standard culture. Of these, 8 were positive for the vanA gene and 4 were positive for the vanB gene, and were later identified as true positives by enriched culture (i.e., a liquid overnight culture, for example, in tryptic soy broth, inoculated with PBS contacted with the swab, and subsequently plated on solid medium containing vancomycin and bile esulin). The overall sensitivity and specificity of this rapid assay is 93.4% and 99.1%, respectively.

Thus, a marked increase in detection sensitivity in comparison to culture was observed using the rapid nucleic acid amplification-based assay described herein. The assay therefore allows the rapid detection of vanA and vanB genes, e.g., in the same day the sample is obtained which is very useful in clinical laboratories and hospitals to identify vancomycin resistance genes. In particular, the method of the invention can identify the vanA or vanB status of a patient and can lead to the appropriate choice of antibiotics to treat an infection, thereby reducing the occurrence of antibiotic resistance. The method also reduces the amount of time a patient has contact with others before their vanA or vanB status is known, and can result in speedier discharge of patients to nursing and extended care facilities, which may require the current vanA or vanB status of those patients.

The invention includes a method to detect the presence of a vanA gene and/or a vanB gene in a biological sample. In one embodiment, the sample is a physiological sample such as a peri-rectal sample. In one embodiment, the sample is from a culture, e.g., a portion of or an individual colony including those from an enriched culture, or from a liquid culture. The method includes providing, e.g., by extracting, nucleic acid from a biological sample of a mammal at risk of having, e.g., by exposure to a mammal having a bacterial infection, or suspected of having a bacterial infection, adding one or more reagents to the nucleic acid sample or a portion thereof, e.g., one or more oligonucleotide primers, to yield an amplification reaction mixture, and subjecting the amplification reaction mixture or a portion thereof to conditions effective to amplify vanA and/or vanB sequences. In one embodiment, the amplification reaction mixture includes two or more oligonucleotide primers specific for one or more different genes in a single reaction vessel. Alternatively, a portion of the nucleic acid sample is added to two or more reaction vessels, and amplification reactions for one or more different genes conducted in those vessels. In one embodiment, separate amplification reactions are conducted, one for the vanA gene and another for the vanB gene. The resulting amplified reactions may then be combined prior to contact with a vanA or vanB-specific probe hybridization. In another embodiment, a single reaction vessel is employed to conduct an amplification reaction for both the vanA gene and the vanB gene.

Optionally, the amplified mixture or the amplification reaction mixture is contacted with at least one probe, e.g., a vanA-specific probe and/or a vanB-specific probe, and optionally one or more reagents, which under appropriate conditions, preferably high stringency conditions, are effective to hybridize a vanA-specific probe and/or a vanB-specific probe to target DNA, i.e., to form a hybrid between the target DNA and sequences in the probe which are complementary thereto, and the presence or amount of hybridized probe detected or determined, e.g., at one or more time points. In one preferred embodiment, the one or more probes are labeled with a detectable moiety or a moiety capable of detection. In one embodiment, a vanA-specific probe is labeled. In another embodiment, a vanB-specific probe is labeled. In another embodiment, a vanA-specific probe and a vanB-specific probe are labeled, e.g., each with a one or more different labels. In one embodiment, the amplified mixture is contacted with at least one probe and one or more reagents, to yield a hybridization reaction mixture. For instance, a portion of the amplification reaction may be added to a reaction vessel and one or more probes and one or more reagents added to the vessel, or a portion of the amplification reaction may be added to at least two reaction vessels and one or more probes and one or more reagents added to each of those vessels. Alternatively, an amplification reaction or a portion thereof is added to one or more probes and one or more reagents in a reaction vessel.

Thus, in one embodiment, the amplification reaction includes a nucleic acid sample and one or more vanA-specific primers and one or more vanB-specific primers, and the resulting amplified mixture is contacted with at least two probes including a vanA-specific probe and a vanB-specific probe under conditions, preferably high stringency conditions, effective to hybridize the probes to their respective target DNAs, i.e., to form a hybrid between the target DNA and sequences in each probe which are complementary thereto. In another embodiment, separate vanA-specific and vanB-specific amplification reactions are conducted, then the reactions or a portion thereof are combined, and a vanA-specific and vanB-specific hybridization reaction conducted in a single vessel. Accordingly, in one embodiment, the method comprises contacting a biological sample comprising nucleic acid or a portion thereof with one or more vanA-specific oligonucleotide primers under conditions effective to amplify vanA sequences. Previously, concurrently or subsequently, e.g., in the same or a different reaction vessel, a nucleic acid sample or a portion thereof is contacted with one or more vanB-specific oligonucleotide primers under conditions effective to amplify vanB sequences. In one embodiment, the amplified sample is contacted with one or more vanA-specific or vanB-specific oligonucleotide probes under high stringency hybridization conditions effective to form a hybrid between the oligonucleotide probes and vanA and/or vanB amplified nucleic acid, and the presence or amount of hybrid formation detected or determined. Hence, in one embodiment, separate vanA-specific and vanB-specific amplification and hybridization reactions are conducted.

In yet another embodiment, the amplification reaction includes a nucleic acid sample, one or more vanA-specific primers and one or more vanB-specific primers, and one or more probes, e.g., at least two probes including a vanA-specific probe and a vanB-specific probe, which is subjected to conditions effective to amplify vanA-specific and vanB-specific sequences and to hybridize the probes to their respective target DNAs. Then the presence or amount of hybrid formation detected or determined. In one preferred embodiment, the one or more probes are labeled with a detectable moiety or a moiety capable of detection. In one embodiment, a vanA-specific probe is labeled. In another embodiment, a vanB-specific probe is labeled. In another embodiment, a vanA-specific probe and a vanB-specific probe are labeled, e.g., each with a one or more different labels. Exemplary conditions for amplification, or amplification and hybridization, include about 55° C. for about 2 minutes, about 95° C. for 10 minutes, followed by about 45 cycles of about 95° C. for about 15 seconds and about 60° C. for about 1 minute. Thus, by probing an amplified sample with probes towards the vanA gene and the vanB gene, a single sample can be employed to detect both antibiotic resistance genes.

In one embodiment, the oligonucleotides of the invention include sequences substantially corresponding to nucleotides 851 to 868 of the vanA gene (SEQ ID NO:2; an exemplary vanA gene has SEQ ID NO:1 from E. faecium pIP816 gi 43335, also see FIG. 1, Accession No. X56895 which corresponds to SEQ ID NO:11), or the complement thereof, or a portion thereof; sequences substantially corresponding to nucleotides 870 to 896 of the vanA gene (SEQ ID NO:3), the complement thereof, or a portion thereof, sequences substantially corresponding to nucleotides 898 to 917 of the vanA gene (SEQ ID NO:4), the complement thereof, or a portion thereof; sequences substantially corresponding to nucleotides 387 to 404 of the vanB gene (SEQ ID NO:6, an exemplary vanB gene has SEQ ID NO:5 which corresponds to Accession No. U00456, also shown in FIG. 2), the complement thereof, or a portion thereof; sequences substantially corresponding to nucleotides 406 to 423 of the vanB gene (SEQ ID NO:7), the complement thereof, or a portion thereof; or sequences substantially corresponding to nucleotides 426 to 446 of the vanB gene (SEQ ID NO:8 or SEQ ID NO:9), the complement thereof, or a portion thereof. In one embodiment, the oligonucleotide primers include sequences substantially corresponding to nucleotides 851 to 868 or 898 to 917 of the vanA gene, the complement thereof or a portion thereof. In one embodiment, the oligonucleotide primers include sequences substantially corresponding to nucleotides 387 to 404 or 426 to 446 of the vanB gene, the complement thereof, or a portion thereof. In one embodiment, the oligonucleotide probe includes sequences substantially corresponding to nucleotides 870 to 896 of the vanA gene, the complement thereof, or a portion thereof, or sequences substantially corresponding to nucleotides 406 to 423 of the vanB gene, the complement thereof, or a portion thereof. Preferably, the probe is labeled, e.g., with one or more labels such as a fluorescent or chemiluminescent label. In one embodiment, the probes for vanA sequences and vanB sequences have different labels. Optionally, one or more non-vanA gene or non-vanB gene probes, may be employed, e.g., to identify the microorganism in the sample and/or to confirm that sufficient DNA was present in the sample to detect the vanA and/or vanB gene (an internal control). In one embodiment, the Tₘ of an oligonucleotide employed as a probe is at least about 10° C. higher than the Tₘ of an oligonucleotide employed as a primer in an amplification/hybridization reaction.

In another embodiment of the invention, one or more vanA-specific and/or vanB-specific oligonucleotide probes are employed with a sample which does not contain amplified nucleic acid. The method includes contacting a sample comprising nucleic acid with at least one vanA-specific oligonucleotide probe and/or at least one vanB-specific oligonucleotide probe under high stringency hybridization conditions effective to form a hybrid between each probe and the target nucleic acid, and the presence or amount of hybrid formation detected or determined. Oligonucleotides useful in this embodiment of the invention include those with sequences substantially corresponding to nucleotides 851 to 917 or any contiguous portion thereof greater than about 15 nucleotides in length, e.g., nucleotides 851 to 868, 870 to 896, or 898 to 917, of the vanA gene, the complement thereof, or a portion thereof, or nucleotides 387 to 446 or any contiguous portion thereof greater than about 15 nucleotides in length, e.g., nucleotides 387 to 404, 406 to 423, or 426 to 446, of the vanB gene, the complement thereof, or a portion thereof.

The invention also includes one or more oligonucleotides. The oligonucleotides include one or more of an oligonucleotide substantially corresponding to nucleotides 851 to 868 of the vanA gene, the complement thereof, or a portion thereof, an oligonucleotide substantially corresponding to nucleotides 898 to 917 of the vanA gene, the complement thereof, or a portion thereof, an oligonucleotide substantially corresponding to nucleotides 870 to 896 of the vanA gene, the complement thereof or a portion thereof, an oligonucleotide substantially corresponding to nucleotides 387 to 404 of the vanB gene, the complement thereof, or a portion thereof, an oligonucleotide substantially corresponding to nucleotides 406 to 423 of the vanB gene, the complement thereof, or a portion thereof, and an oligonucleotide substantially corresponding to nucleotides 426 to 446 of the vanB gene, the complement thereof, or a portion thereof. Each oligonucleotide anneals to vanA and/or vanB DNA under stringent hybridization conditions. In one embodiment, the invention includes an oligonucleotide mix including an oligonucleotide corresponding to nucleotides 851 to 868 of the vanA gene, or a portion thereof, and an oligonucleotide corresponding to the complement of nucleotides 898 to 917 of the vanA gene, or a portion thereof. In one embodiment, the invention includes an oligonucleotide mix including an oligonucleotide corresponding to nucleotides 387 to 404 of the vanB gene, or a portion thereof, and an oligonucleotide corresponding to the complement of nucleotides 426 to 446 of the vanB gene, or a portion thereof.

The invention further includes a probe composition. The composition includes one or more oligonucleotide substantially corresponding to nucleotides 870 to 896 of the vanA gene, the complement thereof, or a portion thereof, or an oligonucleotide substantially corresponding to nucleotides 406 to 423 of the vanB gene, the complement thereof, or a portion thereof.

The invention further includes a kit with primers and/or probes useful to amplify and/or detect the vanA gene and/or the vanB gene in a test sample. The kit includes one or more oligonucleotide comprising sequences corresponding to nucleotides 870 to 896 of the vanA gene, the complement thereof, or a portion thereof, an oligonucleotide comprising sequences corresponding to nucleotides 851 to 868 of the vanA gene, the complement thereof, or a portion thereof, and an oligonucleotide comprising sequences corresponding to nucleotides 898 to 917 of the vanA gene, the complement thereof, or a portion thereof, wherein each oligonucleotide anneals, e.g., under stringent hybridization conditions, to vanA DNA. The kit optionally includes other probes, e.g., non-vanA probes, for instance, primers or probes useful to amplify or detect other genes, including other drug resistance genes.

In one embodiment, the kit includes one or more of an oligonucleotide comprising sequences substantially corresponding to nucleotides 387 to 404 of the vanB gene, the complement thereof, or a portion thereof, an oligonucleotide comprising sequences substantially corresponding to nucleotides 406 to 423 of the vanB gene, the complement thereof, or a portion thereof, or an oligonucleotide comprising sequences substantially corresponding to nucleotides 426 to 446 of the vanB gene, the complement thereof, or a portion thereof. The kit optionally includes other probes, e.g., non-vanB probes, for instance, primers or probes useful to amplify or detect other genes, including other drug resistance genes.

### BRIEF DESCRIPTION OF THE FIGURES

FIG. 1. A representative vanA sequence (SEQ ID NO:1). Underlining shows the position of an exemplary forward primer, probe and reverse primer.

FIG. 2A. Alignment of 8 vanB sequences (SEQ ID NOs:10-16 and 5, respectively), individual sequences (SEQ ID NOs:10-16 and 5), and a consensus (majority) sequence (SEQ ID NO:17).

FIG. 3. Organisms tested for specificity of vanA and vanB primers and probes.

### DETAILED DESCRIPTION OF THE INVENTION

Definitions

As used herein, the following terms have the given meanings unless expressly stated to the contrary.

A “nucleotide” is a subunit of a nucleic acid comprising a purine or pyrimidine base group, a 5-carbon sugar and a phosphate group. The 5-carbon sugar found in RNA is ribose. In DNA, the 5-carbon sugar is 2′-deoxyribose. The term also includes analogs of such subunits, such as a methoxy group (MeO) at the 2′ position of ribose.

An “oligonucleotide” is a polynucleotide having two or more nucleotide subunits covalently joined together. Oligonucleotides are generally about 10 to about 100 nucleotides in length, or more preferably 10 to 50 nucleotides in length. The sugar groups of the nucleotide subunits may be ribose, deoxyribose, or modified derivatives thereof. The nucleotide subunits may be joined by linkages such as phosphodiester linkages, modified linkages or by non-nucleotide moieties that do not prevent hybridization of the oligonucleotide to its complementary target nucleotide sequence. Modified linkages include those in which a standard phosphodiester linkage is replaced with a different linkage, such as a phosphorothioate linkage, a methylphosphonate linkage, or a neutral peptide linkage. Nitrogenous base analogs also may be components of oligonucleotides in accordance with the invention. Ordinarily, oligonucleotides will be synthesized by organic chemical methods and will be single-stranded unless specified otherwise. Oligonucleotides can be labeled with a detectable label.

A “target nucleic acid” is a nucleic acid comprising a target nucleic acid sequence.

A “target nucleic acid sequence,” “target nucleotide sequence” or “target sequence” is a specific deoxyribonucleotide or ribonucleotide sequence that can be hybridized by an oligonucleotide. For instance, a “target nucleic acid sequence region” of bacteria in the Enterococcus genus refers to a nucleic acid sequence present in nucleic acid or a sequence complementary thereto found in Enterococcus bacteria, which is not present in nucleic acids of other species. Nucleic acids having nucleotide sequences complementary to a target sequence may be generated by target amplification techniques such as polymerase chain reaction (PCR).

A “primer” is a single-stranded polyoligonucleotide that combines with a complementary single-stranded target to form a double-stranded hybrid, which primer in the presence of a polymerase and appropriate reagents and conditions, results in nucleic acid synthesis.

A “probe” is a single-stranded polynucleotide that combines with a complementary single-stranded target polynucleotide to form a double-stranded hybrid. A probe may be an oligonucleotide or a nucleotide polymer, and may contain a detectable moiety which can be attached to the end(s) of the probe or can be internal to the sequence of the probe. The nucleotides which combine with the target polynucleotide need not be strictly contiguous as may be the case with a detectable moiety internal to the sequence of the probe.

A “detectable moiety” is a label molecule attached to, or synthesized as part of, a polynucleotide probe. This molecule should be uniquely detectable and will allow the probe to be detected as a result. These detectable moieties include but are not limited to radioisotopes, calorimetric, fluorometric or chemiluminescent molecules, enzymes, haptens, redox-active electron transfer moieties such as transition metal complexes, metal labels such as silver or gold particles, or even unique oligonucleotide sequences.

A “hybrid” is the complex formed between two single-stranded polynucleotide sequences by Watson-Crick base pairings or non-canonical base pairings between the complementary bases. By “nucleic acid hybrid” or “probe:target duplex” is meant a structure that is a double-stranded, hydrogen-bonded structure, preferably 10 to 100 nucleotides in length, more preferably 14 to 50 nucleotides in length. The structure is sufficiently stable to be detected by means such as chemiluminescent or fluorescent light detection, colorimetry, autoradiography, electrochemical analysis or gel electrophoresis. Such hybrids include RNA:RNA, RNA:DNA, or DNA:DNA duplex molecules.

“Hybridization” is the process by which two complementary strands of polynucleotide combine to form a stable double-stranded structure (“hybrid complementarity” is a property conferred by the base sequence of a single strand of DNA or RNA which may form a hybrid or double-stranded DNA:DNA, RNA:RNA or DNA:RNA through hydrogen bonding between Watson-Crick base pairs on the respective strands). Adenine (A) ordinarily complements thymine (T) or uracil (U), while guanine (G) ordinarily complements cytosine (C).

“Stable” means resistant to chemical or biochemical degradation, reaction, decomposition, displacement or modification.

“Stability” means the resistance of a substance to chemical or biochemical degradation, reaction, decomposition, displacement or modification.

The term “stringency” is used to describe the temperature and solvent composition existing during hybridization and the subsequent processing steps. Under high stringency conditions only highly complementary nucleic acid hybrids will form; hybrids without a sufficient degree of complementarity will not form. Accordingly, the stringency of the assay conditions determines the amount of complementarity needed between two polynucleotide strands forming a hybrid. Stringency conditions are chosen to maximize the difference in stability between the hybrid formed with the target and the non-target polynucleotide.

The term “probe specificity” or “primer specificity” refers to a characteristic of a probe or primer which describes its ability to distinguish between target and non-target sequences. Probe or primer specificity is dependent on sequence and assay conditions and may be absolute (i.e., the primer or probe can distinguish between nucleic acid from target organisms and any non-target organisms), or it may be functional (i.e., the primer or probe can distinguish between the nucleic acid from a target organism and any other organism normally present in a particular sample).

“Polynucleotide” means either RNA or DNA, along with any synthetic nucleotide analogs or other molecules that may be present in the sequence and that do not prevent hybridization of the polynucleotide with a second molecule having a complementary sequence. The term includes polymers containing analogs of naturally occurring nucleotides and particularly includes analogs having a methoxy group at the 2′ position of the ribose (MeO).

A “biological sample” refers to a sample of material that is to be tested for the presence of microorganisms or nucleic acid thereof. The biological sample can be obtained from an organism, e.g., it can be a physiological sample, such as one from a human patient, a laboratory mammal such as a mouse, rat, pig, monkey or other member of the primate family, by drawing a blood sample, sputum sample, spinal fluid sample, a urine sample, a rectal swab, a peri-rectal swab, a nasal swab, a throat swab, or a culture of such a sample, e.g., a colony on a plate or a liquid culture. Ordinarily, the biological sample will contain hybridizable polynucleotides. These polynucleotides may have been released from organisms that comprise the biological sample, or alternatively can be released from the organisms in the sample using techniques such as sonic disruption or enzymatic or chemical lysis of cells to release polynucleotides so that they are available for amplification with one or more polynucleotide primers or hybridization with a polynucleotide probe.

“Tₘ” refers to the temperature at which 50% of the probe or primer is converted from the hybridized to the unhybridized form.

One skilled in the art will understand that probes or primers that substantially correspond to a reference sequence or region can vary from that reference sequence or region and still hybridize to the same target nucleic acid sequence. Probes of the present invention substantially correspond to a nucleic acid sequence or region if the percentage of identical bases or the percentage of perfectly complementary bases between the probe and its target sequence is from 100% to 80% or from 0 base mismatches in a 10 nucleotide target sequence to 2 bases mismatched in a 10 nucleotide target sequence. In one embodiment, the percentage is from 100% to 85%. In another embodiment this percentage is from 90% to 100%; and in yet other embodiments, this percentage is from 95% to 100%. Probes or primers that substantially correspond to a reference sequence or region include probes or primers having any additions or deletions which do not prevent the probe or primer from having its claimed property, such as being able to preferentially hybridize under high stringency hybridization conditions to its target nucleic acid over non-target nucleic acids.

By “sufficiently complementary” or “substantially complementary” is meant nucleic acids having a sufficient amount of contiguous complementary nucleotides to form a hybrid that is stable for detection or to initiate nucleic acid synthesis.

By “anti-sense” is meant a nucleic acid molecule perfectly complementary to a reference (i.e., sense) nucleic acid molecule.

“RNA and DNA equivalents” refer to RNA and DNA molecules having the same complementary base pair hybridization properties. RNA and DNA equivalents have different sugar groups (i.e., ribose versus deoxyribose), and may differ by the presence of uracil in RNA and thymine in DNA. The difference between RNA and DNA equivalents do not contribute to differences in substantially corresponding nucleic acid sequences because the equivalents have the same degree of complementarity to a particular sequence.

I. Oligonucleotide Primers and Probes

It is not always necessary to determine the entire nucleic acid sequence of a gene of interest in order to obtain an oligonucleotide primer or probe sequence for that gene or to determine the nucleic acid sequence of that gene from a large number of sources in order to detect heterogenity. Once a sequence is available for a gene of interest or a portion thereof, the following guidelines are useful for designing primers or probes with desired characteristics.

First, the stability of the oligonucleotide:target polynucleotide hybrid is chosen to be compatible with the assay conditions. This may be accomplished by avoiding long A and T rich sequences, by terminating the hybrids with G:C base pairs and by designing the probe in such a way that the Tₘ will be appropriate for standard conditions to be employed in the assay (amplification or hybridization). The nucleotide sequence of the primer or probe should be chosen so that the length and % G and % C result in a probe having a Tₘ about 2 to 10° C. higher than the temperature at which the final assay is performed. The base composition of the primer or probe is significant because G:C base pairs exhibit greater thermal stability when compared with A:T base pairs. Thus, hybrids involving complementary polynucleotides having a high G:C content are generally stable at higher temperatures when compared with hybrids having a lower G:C content.

Second, the position at which the primer or probe binds its target polynucleotide is chosen to minimize the stability of hybrids formed between probe:non-target polynucleotides. This may be accomplished by minimizing the length of perfect complementarity with polynucleotides of non-target organisms, by avoiding G:C rich regions of homology with non-target sequences, and by positioning the primer or probe to span as many destabilizing mismatches as possible. Whether a primer or probe sequence is useful for amplifying or detecting only a specific type of organism or gene depends largely on thermal stability differences between probe:target hybrids and probe:non-target hybrids. The differences in Tₘ should be as large as possible to produce highly specific primers and probes.

The length of the target polynucleotide sequence and the corresponding length of the primer or probe sequence also are important factors to be considered when designing a primer or probe. While it is possible for polynucleotides that are not perfectly complementary to hybridize to each other, the longest stretch of perfectly homologous base sequence is ordinarily the primary determinant of hybrid stability.

Third, regions which are known to form strong internal structures inhibitory to hybridization of a primer or probe are less preferred as targets. Primers or probes having extensive self-complementarity also should be avoided.

Once a presumptive unique sequence has been identified, corresponding oligonucleotides are produced. Defined oligonucleotides that can be used to practice the invention can be produced by any of several well-known methods, including automated solid-phase chemical synthesis using phosphoramidite precursors (Barone et al., 1984). Other well-known methods for construction of synthetic oligonucleotides may, of course, be employed (see Sambrook et al., 1989). All of the oligonucleotides of the present invention may be modified with chemical groups to enhance their performance. Backbone-modified oligonucleotides, such as those having phosphorothioate or methylphosphonate groups, are examples of analogs that can be used in conjunction with oligonucleotides of the present invention. These modifications render the oligonucleotides resistant to the nucleolytic activity of certain polymerases or to nuclease enzymes. Other analogs that can be incorporated into the structures of the oligonucleotides include peptide nucleic acids, or “PNAs.” The PNAs are compounds comprising ligands linked to a peptide backbone rather than to a phosphodiester backbone. Representative ligands include either the four main naturally occurring DNA bases (i.e., thymine, cytosine, adenine or guanine) or other naturally occurring nucleobases (e.g., inosine, uracil, 5-methylcytosine or thiouracil) or artificial bases (e.g., bromothymine, azaadenines or azaguanines, etc.) attached to a peptide backbone through a suitable linker. PNAs are able to bind complementary ssDNA and RNA strands. Methods for making and using PNAs are disclosed in U.S. Pat. No. 5,539,082. Another type of modification that can be used to make oligonucleotides having the sequences described herein involves the use of non-nucleotide linkers (e.g., see U.S. Pat. No. 6,031,091) between nucleotides in the nucleic acid chain which do not interfere with hybridization or optionally elongation of a primer.

Yet other analogs include those which increase the binding affinity of a probe to a target nucleic acid and/or increase the rate of binding of the probe to the target nucleic acid relative to a probe without the analog. Such analogs include those with a modification (substitution) at the 2′ position of a ribofuranosyl nucleotide. Analogs having a modification at the 2′ position of the ribose are one embodiment. Other substitutions at the 2′ position of the sugar are expected to have similar properties so long as the substitution is not so large as to cause steric inhibition of hybridization. Thus, hybridization assay probes can be designed to contain modified nucleotides which, alone or in combination, may have the advantage of increasing the rate of target-specific hybridization.

Preferably, probes are labeled. Essentially any labeling and detection system that can be used for monitoring specific nucleic acid hybridization can be used in conjunction with the probes disclosed herein when a labeled probe is desired. Included among the collection of useful labels are: radiolabels, enzymes, haptens, linked oligonucleotides, colorimetric, fluorometric, e.g., 6-carboxyfluorescein (FAM), carboxytetramethylrhodamine (TAMRA), or VIC (Applied Biosystems), or chemiluminescent molecules, and redox-active moieties that are amenable to electrochemical detection methods. In one embodiment, probes are labeled at one end with a reporter dye and with a quencher at the other end, e.g., reporters including FAM, 6-tetrachlorofluorescein (TET), MAX (Synthegen), Cy5 (Synthegen), 6-carboxy-X-rhodamine or 5(6)-carboxy-X-rhodamine (ROX), and TAMRA and quenchers including TAMRA, BHQ (Biosearch Technologies) and QSY (Molecular Probes). Standard isotopic labels that can be used to produce labeled oligonucleotides include ³H, ³⁵S, ³²P, ¹²⁵I, ⁵⁷Co and ¹⁴C. When using radiolabeled probes, hybrids can be detected by autoradiography, scintillation counting or gamma counting.

Non-isotopic materials can also be used for labeling oligonucleotide probes. These non-isotopic labels can be positioned internally or at a terminus of the oligonucleotide probe. Modified nucleotides can be incorporated enzymatically or chemically with modifications of the probe being performed during or after probe synthesis, for example, by the use of non-nucleotide linker groups. Non-isotopic labels include calorimetric molecules, fluorescent molecules, chemiluminescent molecules, enzymes, cofactors, enzyme substrates, haptens or other ligands. For instance, U.S. Pat. No. 5,998,135 discloses yet another method that can be used for labeling and detecting probes using fluorimetry to detect fluorescence emission from lanthanide metal labels disposed on probes, where the emission from these labels becomes enhanced when it is in close proximity to an energy transfer partner. Exemplary electrochemical labeling and detection approaches are disclosed in U.S. Pat. Nos. 5,591,578 and 5,770,369, and PCT/US98/12082, the disclosures of which are hereby incorporated by reference. Redox active moieties useful as electrochemical labels include transition metals such as Cd, Mg, Cu, Co, Pd, Zn, Fe and Ru. Indeed, any number of different non-isotopic labels can be used for preparing labeled oligonucleotides in accordance with the invention. For example, a probe may contain more than one label.

Alternative procedures for detecting particular genes can be carried out using either labeled probes or unlabeled probes. For example, hybridization assay methods that do not rely on the use of a labeled probe are disclosed in U.S. Pat. No. 5,945,286 which describes immobilization of unlabeled oligonucleotide probe analogs made of peptide PNAs, and detectably labeled intercalating molecules which can bind double-stranded PNA probe/target nucleic acid duplexes. In these procedures, as well as in certain electrochemical detection procedures, such as those disclosed in PCT/US98/12082, PCT/US98/12430 and PCT/US97/20014, the oligonucleotide probe is not required to harbor a detectable label.

Nucleic acid primers and probes specific for a gene of interest, such as a drug resistance gene, optionally in combination with one or more probes specific for a group of organisms, or a universal bacterial probe, find use in an assay to detect the presence of the gene of interest in nucleic acid from a biological sample and optionally to identify a group of organisms and/or to ensure that the nucleic acid in the sample is adequate to detect the gene of interest (i.e., an internal control). For instance, in one embodiment of the invention, a plurality of primers and/or probes specific for the vanA gene and the vanB gene may be employed to detect whether a biological sample contains vanA⁺ or vanA⁻ organisms, as well as vanB⁺ or vanB⁻ organisms.

II. Antibiotic Resistance Gene Primers and Probes

Antimicrobial resistance complicates treatment and often leads to therapeutic failures. Furthermore, overuse of antibiotics inevitably leads to the emergence of bacterial resistance. Besides the rapid identification of negative clinical specimens with DNA-based tests for bacterial detection and the identification of the presence of a pathogen in the positive specimens, the clinician also needs timely information about the ability of the bacterial pathogen to resist antibiotic treatments. Since the sequence from many common bacterial antibiotic resistance genes is available from data banks, the sequence from a portion or from the entire gene is employed to design specific oligonucleotides which will be used as a basis for the development of rapid DNA-based tests.

VanA and vanB sequences and structurally and/or functionally related sequences from a collection of organisms were aligned to identify candidate conserved sequences that could be used to distinguish vanA⁺ and/or vanB⁺ organisms from vanA⁻ and/or vanB⁻ organisms. Thus, by examining partial or complete sequences of vanA⁺ and/or vanB⁺ genes of various organisms, aligning those sequences with structurally and/or functionally related sequences to reveal areas of maximum homology and areas of sequence variation, vanA and/or vanB sequences can be identified that are conserved among vanA and/or vanB genes but exhibit mismatches with structurally and/or functionally related genes. Based on such considerations, the following regions of the vanA gene were chosen to prepare oligonucleotides: nucleotides 851 to 868, nucleotides 870 to 896, and nucleotides 898 to 917 of the vanA gene having SEQ ID NO: 1. Likewise, the following regions of the vanB gene were chosen: nucleotides 387 to 404, nucleotides 406 to 423, and nucleotides 426 to 446 of the vanB gene having SEQ ID NO:5. Such conserved sequences are then tested against a panel of vanA and/or vanB standards and bacterial lysates to verify their utility as primers and/or probes under laboratory conditions. In particular, primers and probes that preferentially anneal to a nucleic acid target region and can initiate nucleic acid synthesis and/or form a detectable duplex that indicates the presence of the vanA gene or vanB gene, are chosen for polynucleotide-based diagnostic assays.

Preferred methods for detecting the presence of the vanA or vanB gene, include the step of contacting a test sample with at least two oligonucleotide primers under conditions that preferentially amplify vanA and/or vanB sequences. Alternatively, a test sample is contacted under high stringency hybridization conditions with at least one oligonucleotide probe that preferentially hybridizes to the vanA and/or vanB gene.

While oligonucleotides probes of different lengths and base composition may be used for detecting the vanA gene or the vanB gene, preferred oligonucleotides have lengths from 15 up to 40 nucleotides and are sufficiently homologous to the target nucleic acid to permit amplification of a vanA or vanB template and/or hybridization to such a template under high stringency conditions. However, the specific sequences described herein also may be provided in a nucleic acid cloning vector or transcript or other longer nucleic acid and still can be used for amplifying or detecting the vanA gene or the vanB gene, i.e., the probes may include sequences unrelated to the vanA or vanB gene, for instance at the 5′ end, the 3′ end, or both the 5′ and 3′ ends. Likewise, primers may include sequences unrelated to the vanA gene and/or the vanB gene, e.g., at the 5′ end. Preferred primers and probes have sequences of up to 40 nucleotides in length and preferably have at least 17 contiguous nucleotides corresponding to sequences in the vanA gene or the vanB gene, or the complement thereof. Preferred oligonucleotide sequences include RNA and DNA equivalents, and may include at least one nucleotide analog.

The primers and probes are tested against synthetic targets as well as tested against biological samples, in an amplification and/or hybridization reaction so as to detect the vanA gene or the vanB gene. In one method of determining whether a biological sample contains vanA or vanB gene sequences, nucleic acids are released from bacterial cells by addition of a lysing agent, e.g., a detergent, or by other known methods for disrupting cells including the use of enzymes, osmotic shock, heat, chemical treatment, and vortexing, for instance, with glass beads, or sonic disruption, for example according to the method disclosed in U.S. Pat. No. 5,374,522. Methods suitable for liberating nucleic acids from cells which can then be subjected to hybridization methods have been described in U.S. Pat. No. 5,837,452 and in U.S. Pat. No. 5,364,763.

Preferably, the probes specifically hybridize to vanA or vanB DNA only under conditions of high stringency. Under these conditions only highly complementary nucleic acid hybrids will form (i.e., those having at least 14 out of 17 bases in a contiguous series of bases being complementary). Hybrids will not form in the absence of a sufficient degree of complementarity. Accordingly, the stringency of the assay conditions determines the amount of complementarity needed between two nucleic acid strands forming a hybrid. Stringency is chosen to maximize the difference in stability between the hybrid formed with target nucleic acid and non-target nucleic acid.

In one embodiment, the vanA oligonucleotides include SEQ ID NOs: 3, 4 or 5, the complement or a portion thereof, and the vanB oligonucleotides include SEQ ID NO:6, 7 or 8, the complement or a portion thereof, which preferentially amplify and/or hybridize to the vanA or vanB gene, respectively.

III. Amplification and Hybridization

Amplification or hybridization assays may be performed either in tubes or in microtitration plates having multiple wells. For assays in plates, the wells may be coated with the specific amplification primers or probes and/or control DNAs, and the detection of amplification products or the formation of hybrids may be automated. Hybridization assays may also be performed on a solid substrate.

A. Amplification

Cells are subjected to conditions which release polynucleotides from the cells, thus forming an extract. For example, cells may be treated with detergents, base and/or heat denatured. If the base is employed, the mixture is then neutralized with an acidic composition. Then reagents are added to yield an amplification reaction (containing, for example, monovalent ions, detergent, dNTPS, primers, and a polymerase).

For DNA amplification by the widely used PCR (polymerase chain reaction) method, primer pairs may be derived from sequenced DNA fragments from clinical samples or from data bank sequences. Prior to synthesis, the potential primer pairs may be analyzed by using the program Oligo™ 4.0 (National Biosciences) to verify that they are likely candidates for PCR amplifications. A select set of primers can then be tested in PCR or other amplification-based assays performed directly from a bacterial suspension or a known standard to determine their specificity.

During DNA amplification by PCR, two oligonucleotide primers binding respectively to each strand of the denatured double-stranded target DNA from the bacterial genome are used to amplify exponentially in vitro the target DNA by successive thermal cycles allowing denaturation of the DNA, annealing of the primers and synthesis of new targets at each cycle (Persing et al, 1993). An exemplary PCR protocols is as follows. Clinical specimens or bacterial colonies are added directly to the 50 μL PCR reaction mixtures containing 50 mM KCl, 10 mM Tris-HCl pH 8.3, 2.5 mM MgCl₂, 0.4 μm of each of the two primers, 200 μM of each of the four dNTPs and 1.25 Units of Taq DNA polymerase (Perkin Elmer). PCR reactions are then subjected to thermal cycling (3 minutes at 95° C. followed by 30 cycles of 1 second at 95° C. and 1 second at 55° C.) using a Perkin Elmer 480™ thermal cycle and subsequently analyzed by standard ethidium bromide-stained agarose gel electrophoresis. It is clear that other methods for the detection of specific amplification products, which may be faster and more practical for routine diagnosis, may be used. Such methods may be based on the detection of fluorescence after amplification (e.g. TaqMan™ system from Perkin Elmer or Amplisensor™ from Biotronics) or other labels such as biotin (SHARP Signal™ system, Digene Diagnostics), or liquid or solid phase hybridization with an oligonucleotide probe binding to internal sequences of the specific amplification product, e.g., a labeled probe. Methods based on the detection of fluorescence are very rapid and quantitative, and can be automated. For instance, one of the amplification primers or an internal oligonucleotide probe specific to the amplicon(s) is coupled with the fluorochrome or with any other label. Moreover, methods based on the detection of fluorescence are particularly suitable for diagnostic tests since they are rapid and flexible as fluorochromes emitting different wavelengths are available (Perkin Elmer). Further, a variety of fluorochromes emitting at different wavelengths, each coupled with a specific oligonucleotide linked to a fluorescence quencher which is degraded during amplification, thereby releasing the fluorochrome (e.g., TaqMan™, Perkin Elmer), may be employed.

To assure PCR efficiency, glycerol or dimethyl sulfoxide (DMSO) or other related solvents, can be used to increase the sensitivity of the PCR and to overcome problems associated with the amplification of target with a high GC content or with strong secondary structures. The concentration ranges for glycerol and DMSO are 5 to 15% (v/v) and 3 to 10% (v/v), respectively. For the PCR reaction mixture, the concentration ranges for the amplification primers and the MgCl₂ are about 0.1 to 1.0 and 1.5 to 3.5 mM, respectively. Modifications of the standard PCR protocol using external and nested primers (i.e., nested PCR) or using more than one primer pair (i.e., multiplex PCR) may also be used (Persing et al, 1993), for instance, to detect simultaneously several genes, including antibiotic resistance genes and genes useful to identify species of bacterial pathogens.

The person skilled in the art of DNA amplification knows the existence of other rapid amplification procedures which include linear amplification procedure, e.g., ligase chain reaction (LCR), transcription-based amplification systems (TAS), self-sustained sequence replication (3SR), nucleic acid sequence-based amplification (NASBA), strand displacement amplification (SDA) and branched DNA (bDNA) (Persing et al, 1993). The scope of this invention is not limited to the use of amplification by PCR, but rather includes the use of any rapid nucleic acid amplification methods or any other procedures which may be used to increase rapidity and sensitivity of the tests. Any oligonucleotides suitable for the amplification of specific nucleic acid sequences by approaches other than PCR and within scope of this invention.

Standard precautions to avoid false positive PCR results should be taken. Methods to inactivate PCR amplification products such as the inactivation by uracil-N-glycosylase may be used to control PCR carryover. For example, in the case of direct amplification from a colony, a portion of the colony may be transferred directly to a 50 μL PCR reaction mixture (e.g., containing 50 mM KCl, 10 mM Tris pH 8.3, 2.5 mM MgCl₂, 0.4 μM of each of the two primers, 200 μM of each of the four dNTPs and 1.25 Unit of Taq DNA polymerase (Perkin Elmer)) using a plastic rod. For the bacterial suspension, 4 μL of a cell suspension may be added to 46 μL of the same PCR reaction mixture. For both strategies, the reaction mixture is overlaid with 50 μL of mineral oil and PCR amplifications are carried out for instance using an initial denaturation step of 3 minutes at 95° C. followed by 30 cycles consisting of a 1 second denaturation step at 95° C. and of a 1 second annealing step at 55° C. in a Perkin Elmer 480™ thermal cycler. PCR amplification products can be analyzed by standard agarose gel (2%) electrophoresis. Amplification products are visualized in agarose gels containing 2.5 μg/mL of ethidium bromide under UV at 254 nm. The entire PCR assay can be completed in approximately one hour.

Alternatively, amplification from bacterial cultures may be performed as described above but using a “hot start” protocol. In that case, an initial reaction mixture containing the target DNA, primers and dNTPs was heated to about 85° C. prior to the addition of the other components of the PCR reaction mixture. The final concentration of all reagents was as described above. Subsequently, the PCR reactions were submitted to thermal cycling and analysis as described above.

To improve bacterial cell lysis and eliminate the PCR inhibitory effects of clinical specimens, samples may be diluted in lysis buffer containing detergent(s). Subsequently, the lysate is added directly to the PCR reaction mixture. Heat treatments of the lysates, prior to DNA amplification, using the thermocycler or a microwave oven may also be performed to increase the efficiency of cell lysis. PCR has the advantage of being compatible with crude DNA preparations. Thus, samples such as blood, cerebrospinal fluid and sera may be used directly in PCR assays after a brief heat treatment.

B. Hybridization

In hybridization experiments, oligonucleotides (of a size less than about 100 nucleotides) have some advantages over DNA fragment probes of greater than 100 nucleotides in length for the detection of bacteria such as ease of preparation in large quantities, consistency in results from batch to batch and chemical stability. The oligonucleotide probes may be derived from either strand of the target duplex DNA. The probes may consist of the bases A, G, C, or T or analogs thereof. In one embodiment, the target DNA is denatured, fixed onto a solid support and hybridized with a DNA probe. Conditions for pre-hybridization and hybridization can be as follows: (i) pre-hybridization in 1 M NaCl+0% dextran sulfate+1% SDS (sodium dodecyl sulfate)+1 μg/ml salmon sperm DNA at 65° C. for 15 minutes, (ii) hybridization in fresh pre-hybridization solution containing the labeled probe at 65° C. overnight, and (iii) post-hybridization including washing twice in 3×SSC containing 1% SDS (1×SSC is 0.15 M NaCl, 0.015 M NaCitrate) and twice in 0.1×SSC containing 0.1% SDS; all washes at 65° C. for 15 minutes. For probes labeled with radioactive labels, the detection of hybrids is preferably by autoradiography. For non-radioactive labels, such as probes having colorimetric, fluorescent or chemiluminescent labels, target DNA need not be fixed onto a solid support.

For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% sodium dodecylsulfate (SDS), and 10% dextran sulfate at 42° C., with washes at 42° C. in 0.2×SSC and 0.1% SDS. Exemplary low stringency conditions include hybridization with a buffer solution of 30 to 35% foarnamide, 1 M NaCl, 1% SDS (sodium dodecyl sulphate) at 37° C., and a wash in 1× to 2×SSC (20×SSC=3.0 M NaCU/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3.

Results from an amplification and/or probe hybridization reaction can be inputted into a computer or data processor (“computer”), either manually using a keyboard or directly through an interface from an automated device such as a plate reader, film scanner or luminometer. The computer can sort the positive and negative results for a particular sample to establish a profile be compared with a look-up table stored in a memory device linked to the computer to associate the profile with results obtained using control organisms in order to determine the presence or absence of a gene of interest in the test organism. Thus, one or more vanA or vanB probes can be used to identify the vanA or vanB status of a sample. Of course, a series of positive and negative control hybridizations can be carried out in parallel to ensure validity of the testing results.

IV. Kits of the Invention

A test kit may contain one or more oligonucleotides of the invention, e.g., one or more primers or one or more probes specific for one or more antibiotic resistance genes, e.g., the vanA or vanB gene, and optionally for particular species of bacterium as well as control primers or probes. The kit is provided in the form of test components and, if present, the probe may be unlabeled or labeled, e.g., labeled with a non-radioactive label. Preferably, if more than one labeled probe is present, each is labeled with a different label. The kit will also optionally include test reagents necessary to perform the amplification reaction, e.g., a polymerase, dNTPs, one or more salts, and/or a buffer, and/or reagents necessary to perform the hybridization reaction, e.g., reagents for pre-hybridization, hybridization, washing steps and/or hybrid detection. The kit may include standard samples to be used as negative and positive controls for each test.

In one embodiment, a test kit includes all reagents and controls to perform DNA amplification assays. Diagnostic kits are adapted for amplification by PCR (or other amplification methods) performed directly either from clinical specimens, or from a bacterial colony. Components required for detection of antibiotic resistance genes, and bacterial identification may be included.

It is understood that the use of the probes and amplification primers described in this invention for bacterial detection and identification is not limited to clinical microbiology applications. In fact, these tests could be used by industries for quality control of food, water, pharmaceutical products or other products requiring microbiological control. These tests could also be applied to detect and identify bacteria in biological samples from organisms other than humans (e.g. other primates, mammals, farm animals and live stocks). These diagnostic tools could also be very useful for research purposes including clinical trials and epidemiological studies.

V. Apparatus Useful for Conducting Hybridization Reactions

Examples of formats that can be used to conduct hybridization reactions include, but are by no means limited to: individual tubes each with a different probe or comprising a plurality of probes; the wells of a 96-well or other multi-well microtiter plate; and a solid support such as a dipstick or a “DNA chip” where polynucleotide probes are immobilized to the support at different addresses in a spaced-apart configuration. Identifying microorganisms and/or the presence of gene(s) of interest advantageously can be performed without requiring any in vitro amplification step. Alternatively, an amplification step, may be employed.

According to one approach for conducting hybridization procedures, probes can be labeled with distinguishable labels. More particularly, a single tube, well, or support may include distinct probes that are independently labeled with labels that emit peak energy at different times after generating a light emission. Materials and methods that can be used for making and using distinguishable probes useful in connection with the present invention can be found in U.S. Pat. No. 5,756,011. Fluorescent labels that produce light at different wavelengths following excitation represent still other examples of distinguishable labels that can be used in connection with the procedures described herein. In this way, two probes that employ distinguishable labels can be distinguished from each other even though they are combined at the same locus of a testing device. Accordingly, it is possible to combine large numbers of different probes at a single location while still being able to distinguish the results of hybridization for the different probes or sets of probes.

In one embodiment, at least two probes in a single hybridization reaction are labeled with detectable moieties which are distinguishable. The labeled probes are mixed and allowed to hybridize to any nucleic acid contained in the test sample having a sequence sufficiently complementary to the probe sequence to allow hybridization under appropriately selective conditions. One labeling reagents are particularly useful in, although not limited to, a homogeneous assay system in which the presence and quantification of the analytes of interest may be detected and measured without the need for the analyte-bound label to be physically separated from the unbound label prior to detection. However, such reagents may be used in heterogeneous systems or in combinations of homogeneous and heterogenous assay systems as well.

The compositions and methods provided herein may be utilized in a wide variety of other/related methods (e.g., U.S. Pat. Nos. 5,210,015; 5,487,972; 5,422,253; 5,691,142; 5,719,028; 5,130,238; 5,409,818; 5,554,517; 5,589,332, 5,399,491; 5,480,784; 5,215,899; 5,169,766; 5,194,370; 5,474,916; 5,698,400; 5,656,430; and PCT publication nos. WO 88/10215; WO 92/08800, WO 96/02668; WO 97/19193; WO 97/09444; WO 96/21144; WO 92/22671). Other variations of this assay include ‘exponential’ cycling reactions such as described in U.S. Pat. No. 5,403,711 (see also U.S. Pat. No. 5,747,255).

Representative examples of further suitable assay formats including any of the above assays which are carried out on solid supports such as dipsticks, magnetic beads, and the like (see generally U.S. Pat. Nos. 5,639,428; 5,635,362; 5,578,270; 5,547,861; 5,514,785; 5,457,027; 5,399,500; 5,369,036; 5,260,025; 5,208,143; 5,204,061; 5,188,937; 5,166,054; 5,139,934; 5,135,847; 5,093,231; 5,073,340; 4,962,024; 4,920,046; 4,904,583; 4,874,710; 4,865,997; 4,861,728; 4,855,240; and 4,847,194).

The invention will be further described by the following non-limiting example.

### EXAMPLE 1

Materials and Methods

Oligonucleotides

Primer Sequences

vanA forward primer: CCG GTG GCA GCT ACG TTT (SEQ ID NO:2) (61% GC content).

vanA reverse primer: CAC CGA AGG ATG AGC CTG AA (SEQ ID NO:4) (55% GC content).

vanA probe: CCT ATC CTG TTT TTG TTA AGC CGG CGC (SEQ ID NO:3, labeled at the 5′ end with 6-FAM and at the 3′ end with TAMRA) (57% GC content).

The vanA amplicon has a length of 67 bp, a Tₘ of 82° C., 55% GC content, and a Tₐ of 60.

vanB forward primer: CGA CCT CAC AGC CCG AAA (SEQ ID NO:6).

vanB reverse primer: CGG CAG GAC AAT ATG ATG GAA (SEQ ID NO:8), or CAG CAG GAC AAT ATG ATG GAA (SEQ ID NO:9).

vanB probe: CGC TTG CTC AAT TAA GAT (SEQ ID NO:7, labeled at the 5′ end with VIC and at the 3′ end with a non-flourescent quencher MGB).

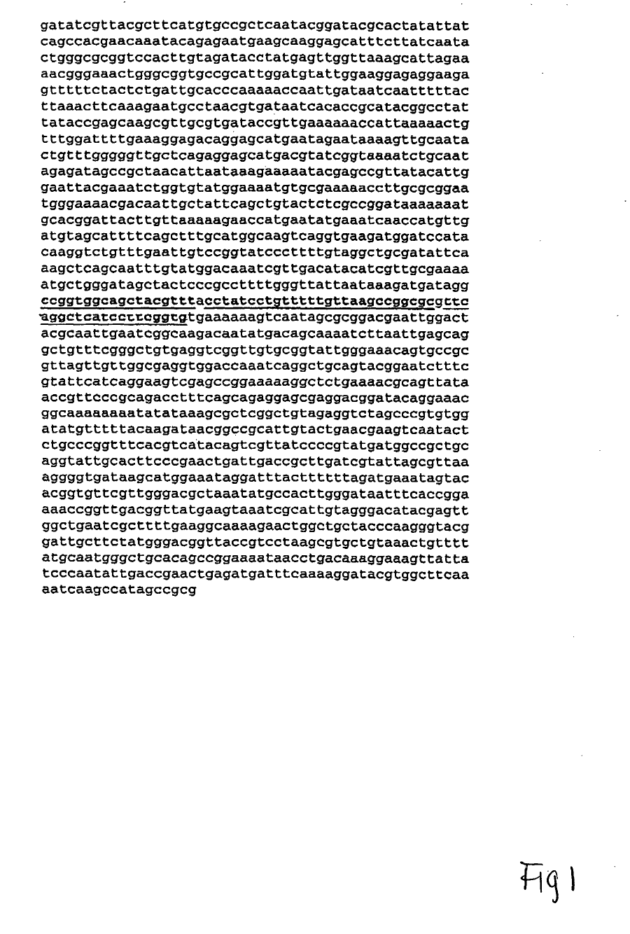

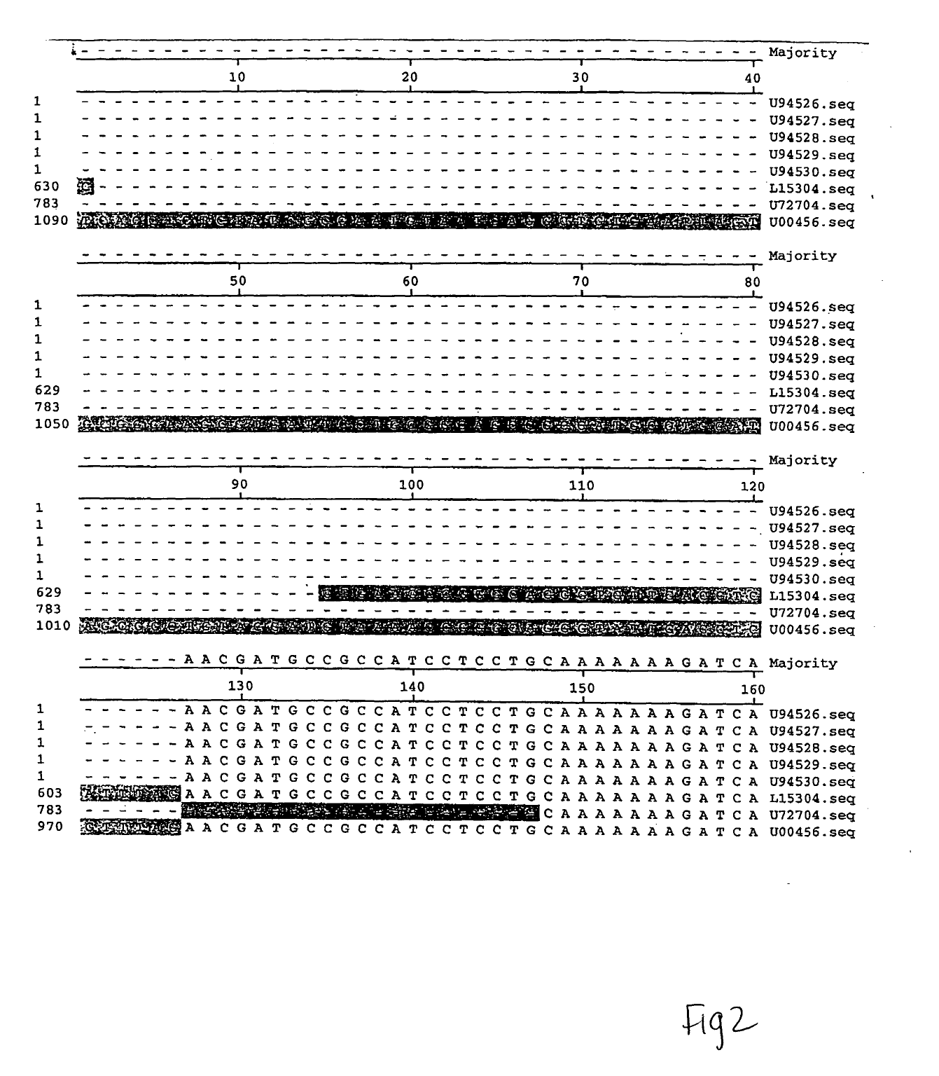

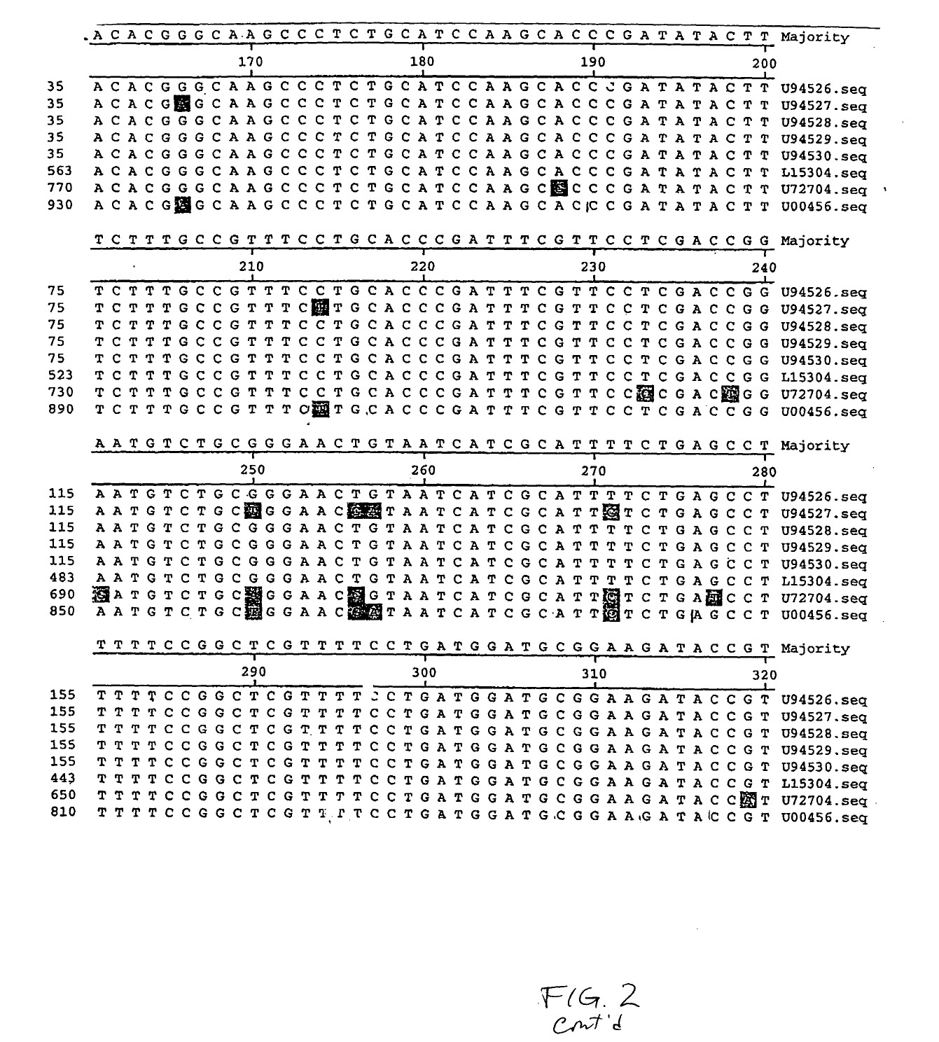

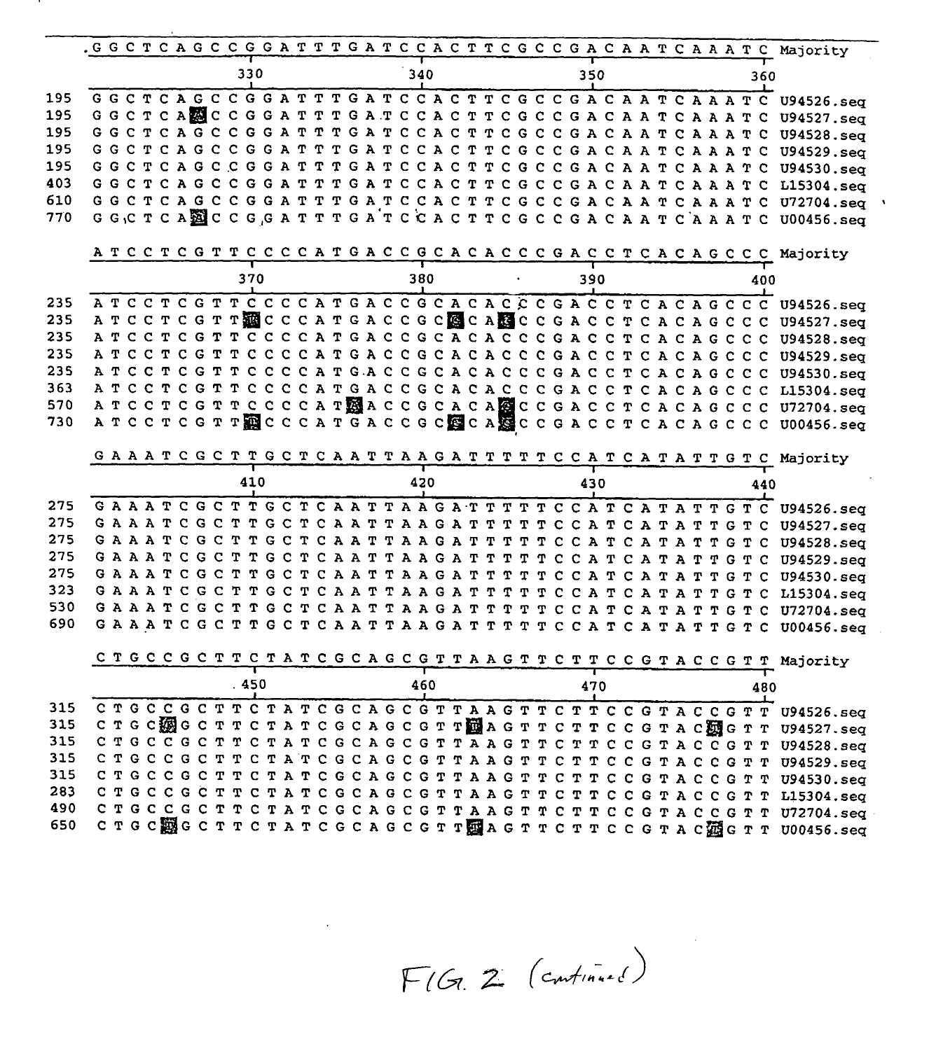

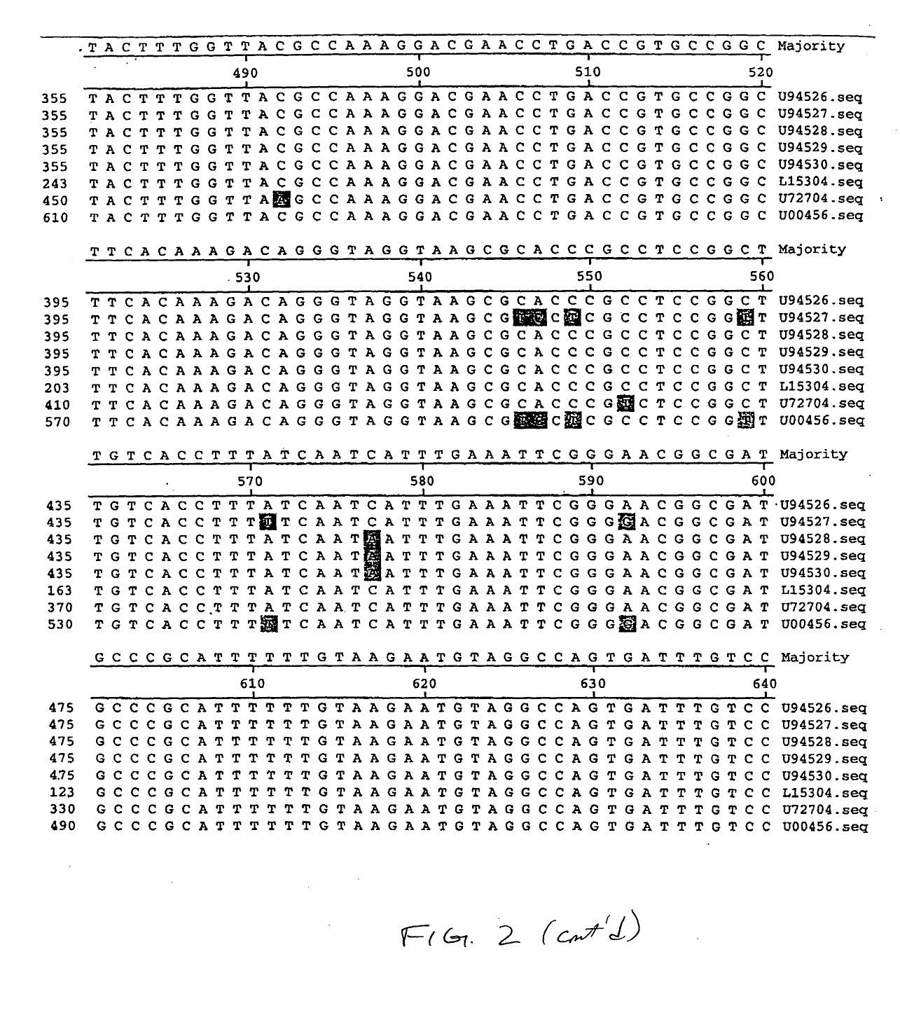

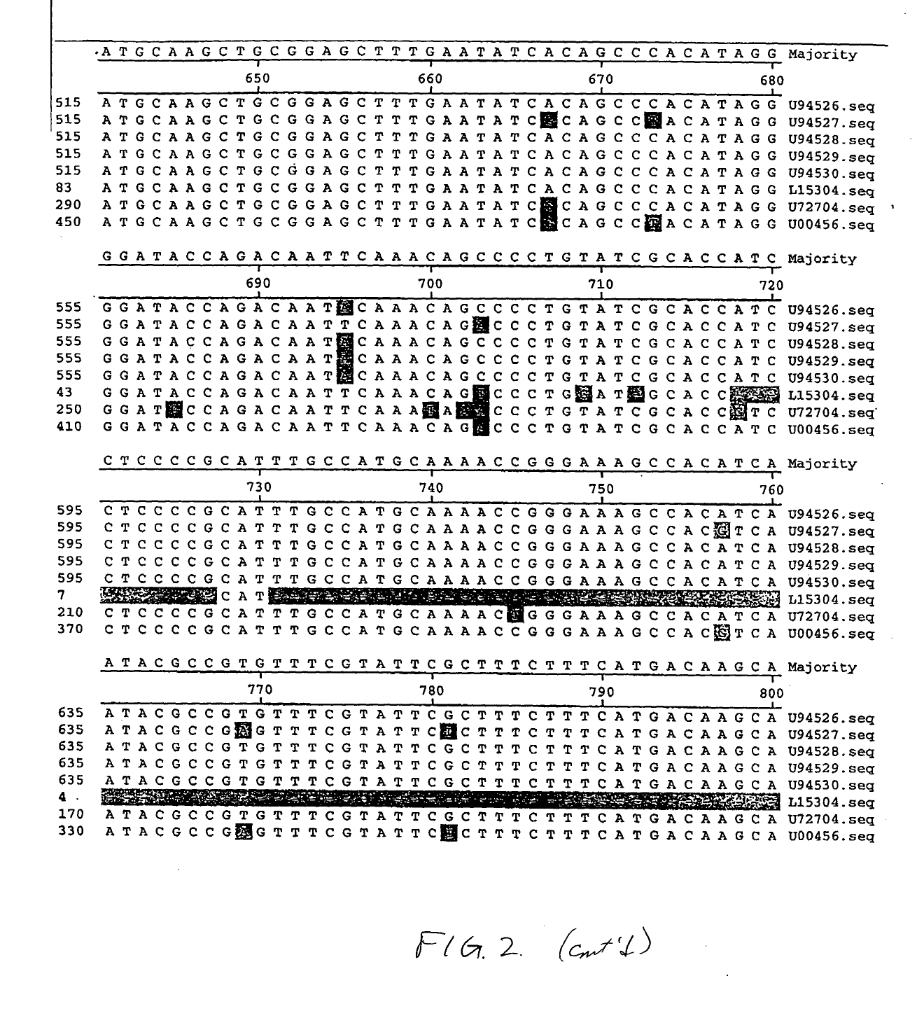

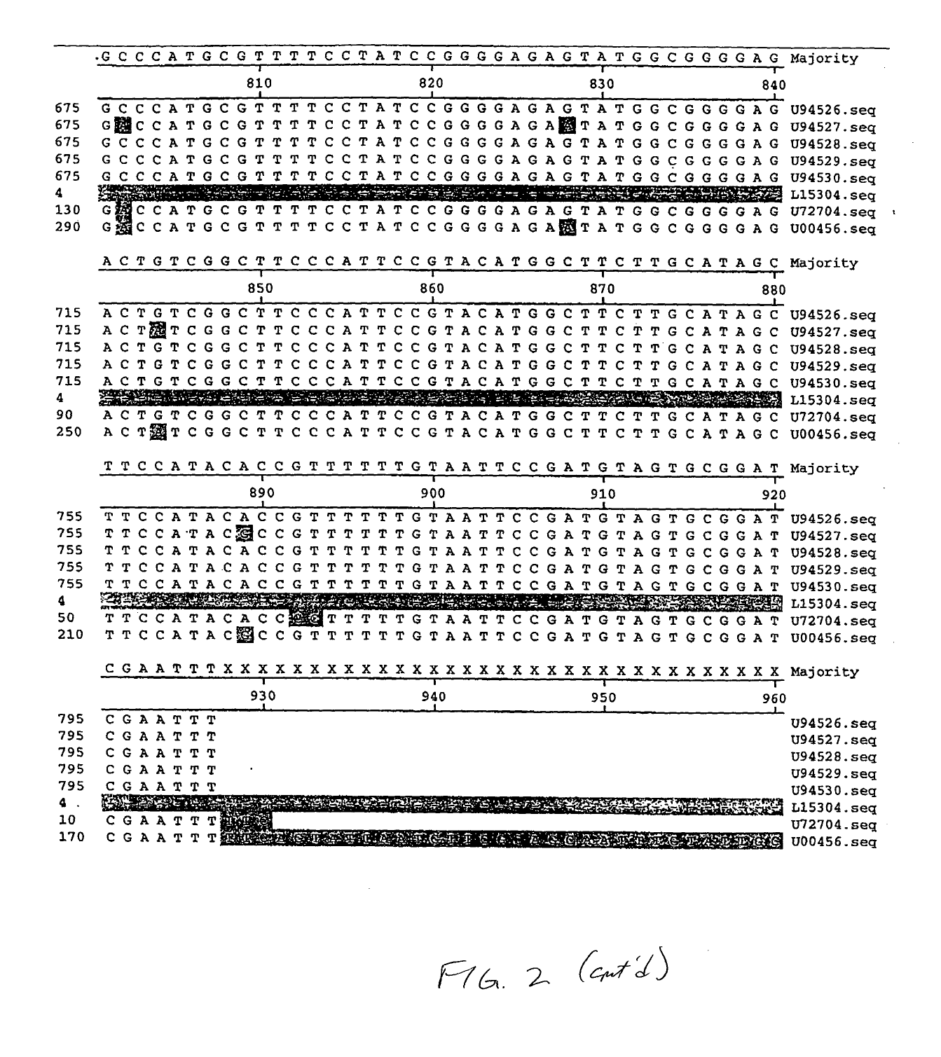

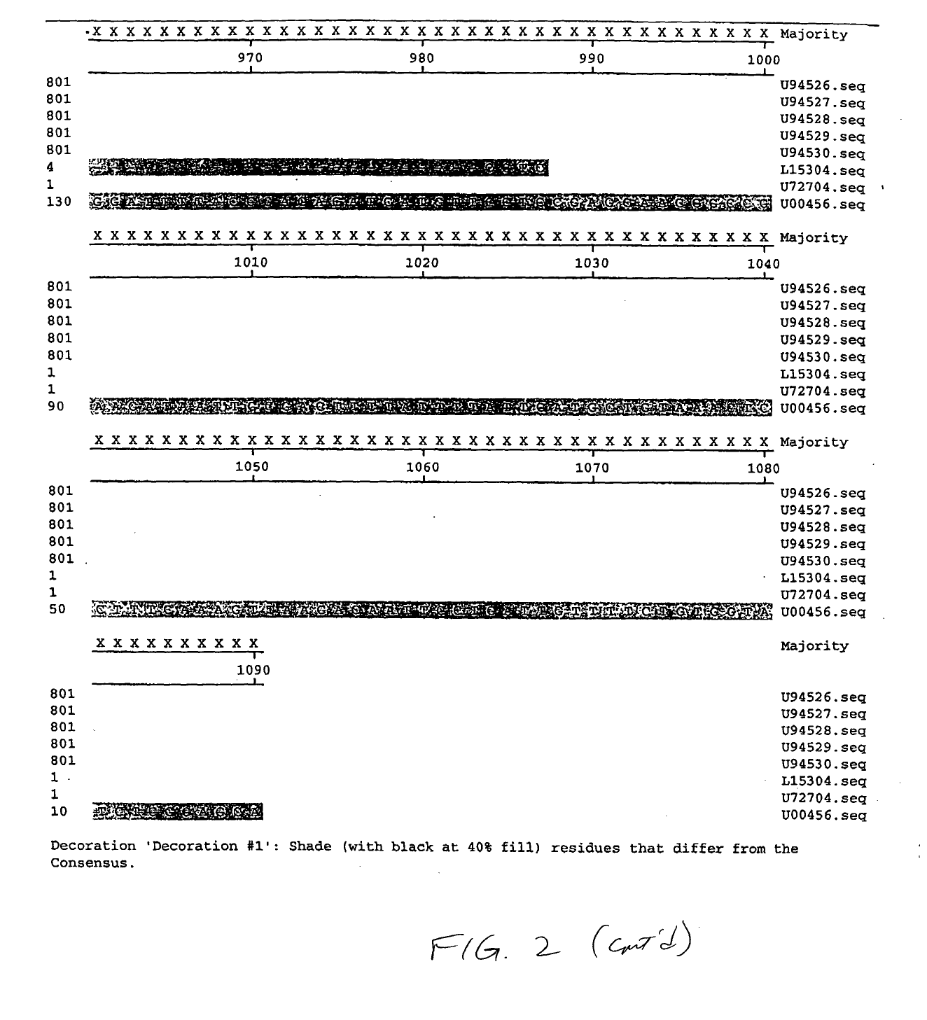

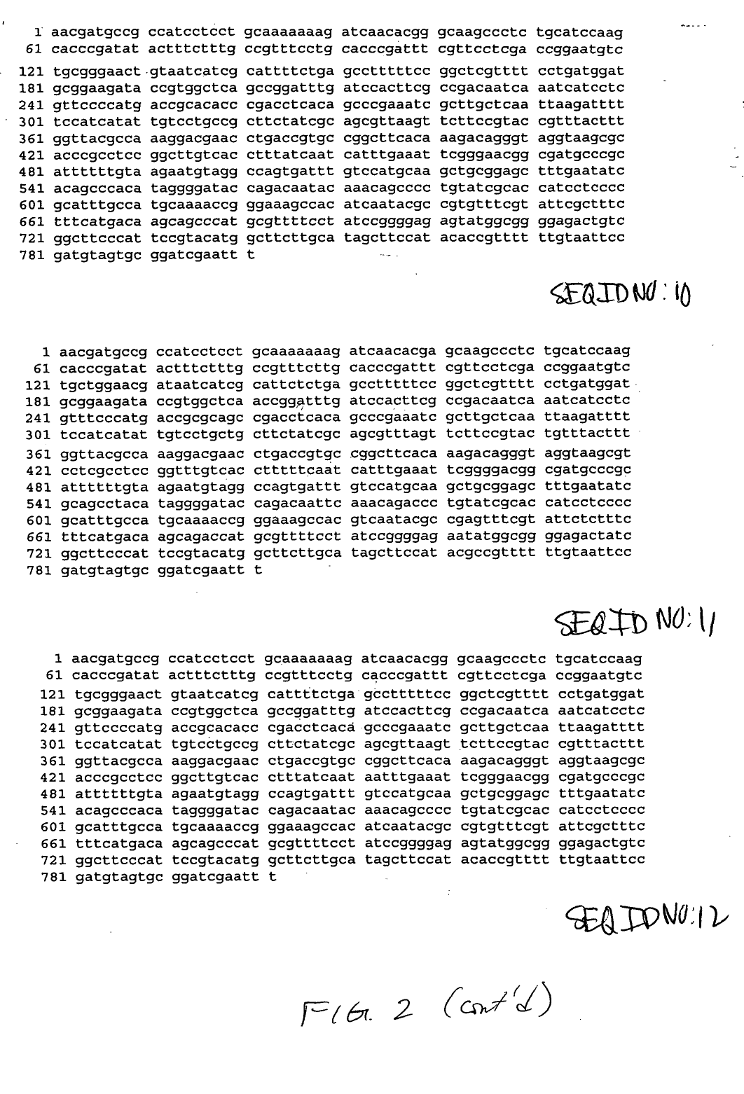

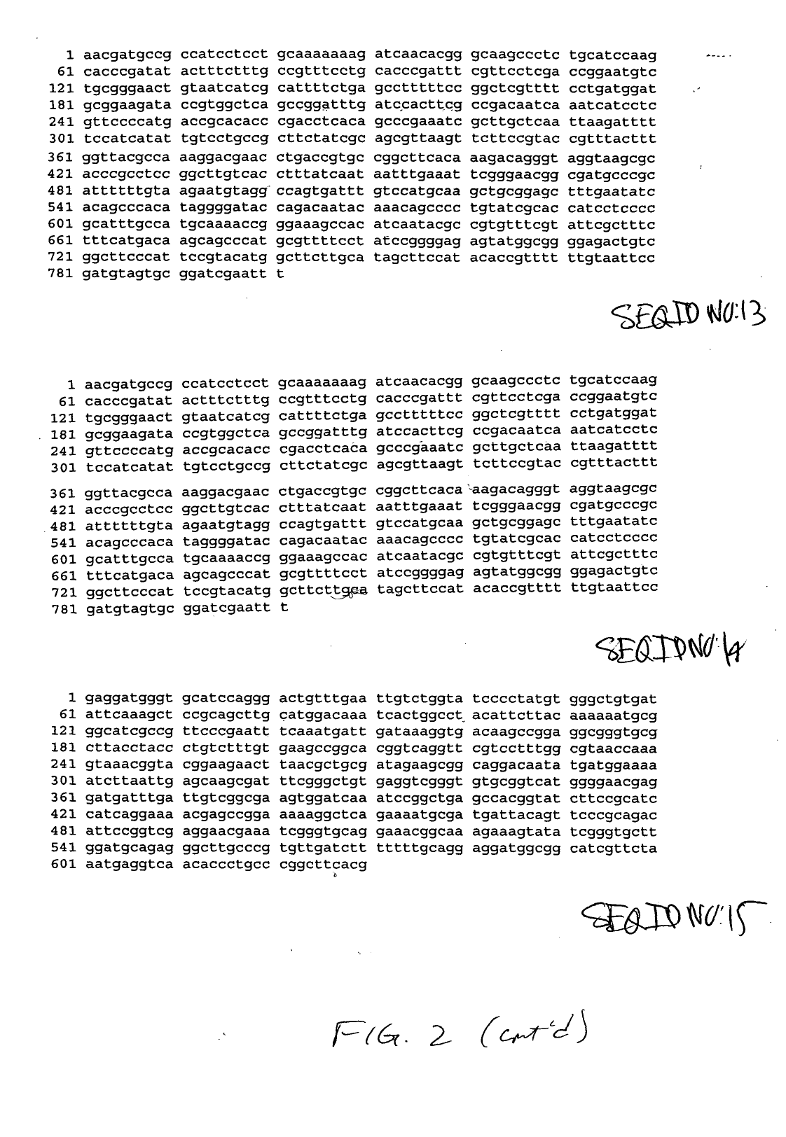

The sequence for the vanA primers was based on the vanA gene sequence from GenBank E. faecium pIP816 gi 43335 (FIG. 1). The sequence for the vanB primers was based on a conserved region found in an alignment of 8 known clinical sequences (FIG. 2). Generally, oligonucleotide criteria were selected as follows: minimum of 30% and maximum of 80% GC content, preferably about 50% GC content, no repeats, no GC rich 3′ end, about 15 to 20 contiguous nucleotides of vanA or vanB-specific sequences, Tₘ of about 59° C., and a maximum 3′ consensus match of 7 nucleotides. All sequences selected were then run through BLAST to ensure that there was no cross reactivity with other organisms.

Sample Processing and Reaction Conditions

Controls included a 500 μl negative extraction control (sterile RNA/DNA free water) and a 500 μl positive extraction control for each of a vanA and a vanB bacterial suspension, optionally run in duplicate, a no template control, optionally in duplicate for each set of primers, and optionally an internal control (e.g., using the ABI internal control kit).

Precautions to limit false positives were employed, e.g., the use of separate work areas, dedicated equipment and lab coats, and decontamination, e.g., 10% bleach, sanicloth disinfectant, and UV.

Primers were diluted to 2 μM and probes to 1 μM with sterile RNA/DNA free water. Patient samples were processed before the positive extraction control then the reagent blank was processed. Each patient swab was introduced to a tube with 1 ml PBS, then vortexed. The swab was removed and sediment allowed to form for 5 minutes at room temperature. The samples can be stored at 2-8° C. for up to 7 days. 500 Al of a cell lysis solution was added to 500 μl of a patient sample in an eppendorf tube, vortexed, then incubated at 65° C. for 15 minutes in a dry heat block. 200 μl of a protein precipitation solution was added to each tube, and vortexed, after which the sample was placed on ice for 5 minutes. The sample was subjected to centrifugation at 14,000×g for 3 minutes. The supernatant was added to a fresh tube containing 600 μl of isopropanol. The tube was inverted several times, then incubated at room temperature for 5 minutes. The mixture was subjected to centrifugation at 14,000×g for 5 minutes, the resulting supernatant discarded, and residual liquid drained. 600 μl of 70% ethanol was added to the pellet, the tube inverted several times, and subsequently subjected to centriftigation at 14,000×g for 1 minute. The supernatant was discarded and the pellet dried. The dried pellet was resuspended in 20 μl of sterile RNA/DNA free water and stored at 2-8° C. or less than 0° C.

For each reaction, the following reagents were added and mixed.

| | |
|---|---|
| 12.5 μl | 2X ABI Master mix |
| 3.0 μl | sterile water |
| 2.5 μl | forward primer |
| 2.5 μl | reverse primer |
| 2.5 μl | probe |
| 2 μl | sample |
| 25 μl | |

For reaction mixtures for multiple samples, 23 μl of a reaction mixture (without added sample) was added to each reaction vessel, e.g., one or more wells of a 96-well plate, then 2 μl of a control sample or a DNA sample added. The reaction vessels were then sealed, e.g., by sealing the 96-well plate. Assay conditions included about 55° C. for about 2 minutes, about 95° C. for 10 minutes, followed by about 45 cycles of about 95° C. for about 15 seconds and about 60° C. for about 1 minute.
Results

Currently the gold standard for the detection of VRE is culture. This not only lacks sensitivity but also is time consuming. Time is key in that hospital cost is increased whilst patient status is being determined and that in this time, infected patients can potentially spread the organism to other patients. In particular, knowledge of whether patients carry vancomycin resistance genes is paramount in high-risk units and long term care facilities. Prevention of spread is the key as the resistance genes may be transferred to another bacterium, e.g., methicillin resistant Staphylococcus aureus (MRSA), an organism that is currently sensitive to vancomycin. If MRSA acquires this resistance mechanism, there are very few treatments left for that particularly virulent organism.

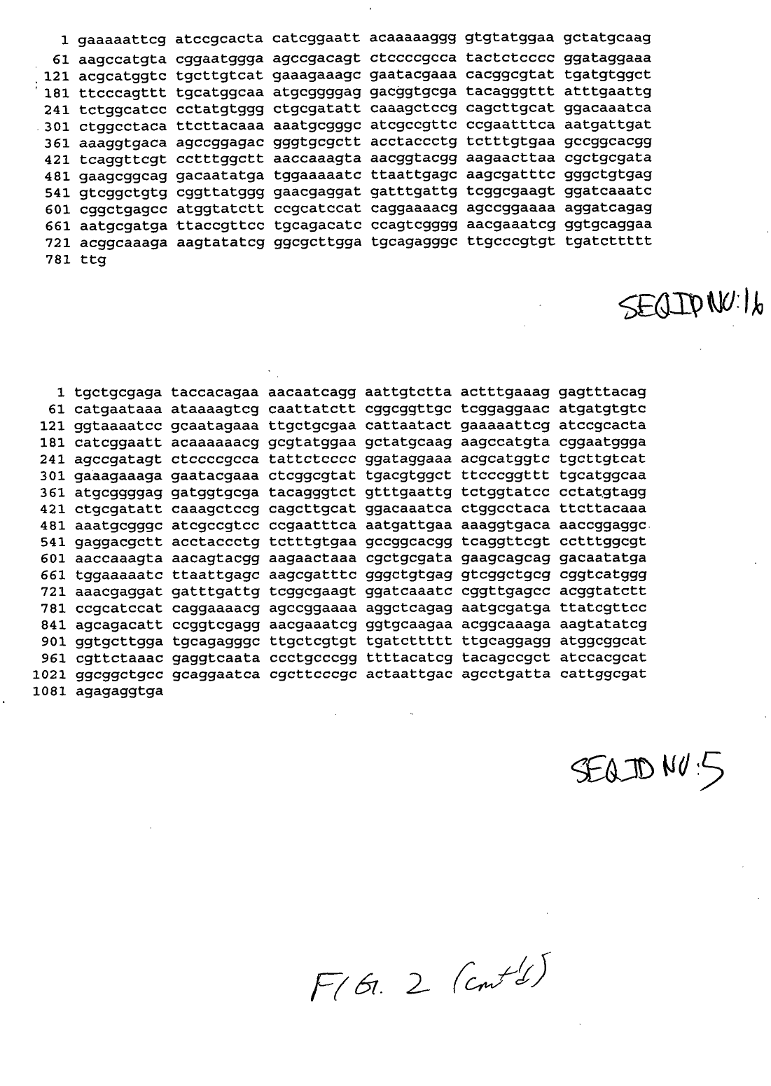

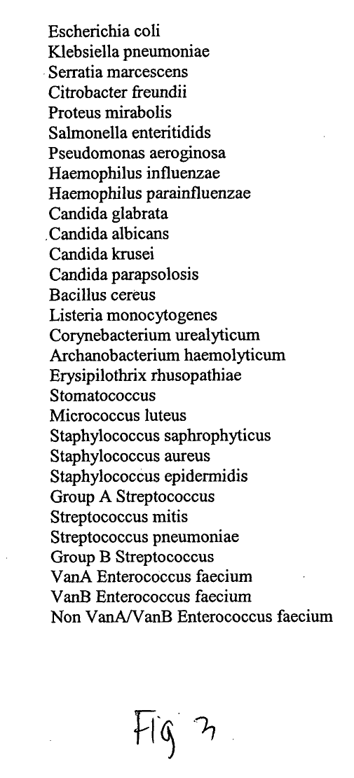

Primers were employed to amplify vanA and vanB resistance genes in Enterococci from peri-rectal swabs, and probes were employed to detect vanA and vanB resistance genes. 305 samples were tested in total, and the results compared to culture directly in samples after they were routinely processed. Using real time PCR, vancomycin resistance genes, vanA and vanB, were amplified from vancomycin resistant enterococci (VRE) directly from a peri-rectal swab. The real time PCR assay resulted in a sensitivity of 93.4% and a specificity of 99.1% (true positives 73, true negatives 224, false negatives 6, false positives 2). Thirty other lab organisms including those that reside in the gut (FIG. 3) were tested with the primers and none of them were positive, thus demonstrating specificity. Therefore, such an assay can be used clinically as a diagnostic test and can yield a result the same day as the sample is obtained. Moreover, the assay is considerably more sensitive in detecting patients missed by culture.

Hence, the assay described herein overcomes the long time and low sensitivity of the current method used clinically to detect VRE. In addition, the primer and probe sets described herein to amplify and detect the vanA and vanB genes result in high sensitivity and specificity.

References

Arthur et al., J. Bacteriol., 174:2582 (1992).

Barone et al., Nucl. Acids Res., 12:4051 (1984).

Bemston et al., LPTP Newsletter, 221:1 (1998).

Centers for Disease Control and Prevention, Morbidity and Mortality Weekly Report, 42:597 (1993).

Centers for Disease Control and Prevention, Morbidity and Mortality Weekly Report, 44:1 (1995).

Evers et al., Microbiol Drug Resistance, 2:219 (1996).

Murray, Clin. Microbiology Rev., 3:46 (1990).

Persing et al, 1993. Diagnostic Molecular Microbiology: Principles and Applications, American Society for Microbiology, Washington, D.C.

Petrich e al., Molecular and Cellular Probes, 13:275 (1999).

Petrich et al., Diag. Micro. Infect. Dis., 41:215 (2001).

Quintiliani et al., Gene, 172:1 (1996).

Sahm et al., J. Clin. Microbiol., 35:2026 (1997).

Sambrook et al., Molecular Cloning: A Laboratory Manual, 11 (1989).

Satake et al., J. Clin. Microbiol., 35:2325 (1997).

Van Horn et al., J. Clin. Microbiol., 34:924 (1996).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to detect vanA in a sample, comprising:
a) contacting a sample suspected of comprising amplified vanA nucleic acid with at least one vanA-specific oligonucleotide probe under high stringency hybridization conditions effective to form a hybrid between the vanA-specific oligonucleotide probe and vanA nucleic acid in the sample, wherein the vanA-specific oligonucleotide probe comprises sequences which include sequences substantially corresponding to nucleotides 870 to 896 of the vanA gene, the complement thereof, or a portion thereof, sequences substantially corresponding to nucleotides 851 to 868 of the vanA gene, the complement thereof, or a portion thereof, or sequences substantially corresponding to nucleotides 898 to 917 of the vanA gene, the complement thereof, or a portion thereof; and
b) detecting or determining the presence or amount of hybrid formation.

2. A method to detect vanB in a sample, comprising:
a) contacting a sample suspected of comprising amplified vanB nucleic acid with at least one vanB-specific oligonucleotide probe under high stringency hybridization conditions effective to form a hybrid between the vanB-specific oligonucleotide probe and vanB nucleic acid in the sample, wherein the vanB-specific oligonucleotide probe comprises sequences which include sequences substantially corresponding to nucleotides 387 to 404 of the vanB gene, the complement thereof, or a portion thereof, sequences substantially corresponding to nucleotides 406 to 423 of the vanB gene, the complement thereof, or a portion thereof, or sequences substantially corresponding to nucleotides 426 to 446 of the vanB gene, the complement thereof, or a portion thereof; and
b) detecting or determining the presence or amount of hybrid formation.

3. A method to detect vanA in a sample, comprising:
a) contacting a biological sample suspected of comprising nucleic acid with at least one vanA-specific oligonucleotide primer under conditions effective to amplify vanA nucleic acid, wherein the vanA-specific oligonucleotide primer comprises sequences which include sequences substantially corresponding to nucleotides 870 to 896 of the vanA gene, the complement thereof, or a portion thereof, sequences substantially corresponding to nucleotides 851 to 868 of the vanA gene, the complement thereof, or a portion thereof, or sequences substantially corresponding to nucleotides 898 to 917 of the vanA gene, the complement thereof, or a portion thereof, and
b) detecting or determining the presence or amount of amplified nucleic acid.

4. A method to detect vanB in a sample, comprising:
a) contacting a biological sample suspected of comprising nucleic acid with at least one vanB-specific oligonucleotide primer under conditions effective to amplify vanB nucleic acid, wherein the vanB-specific oligonucleotide primer comprises sequences which include sequences substantially corresponding to nucleotides 387 to 404 of the vanB gene, the complement thereof, or a portion thereof, sequences substantially corresponding to nucleotides 406 to 423 of the vanB gene, the complement thereof, or a portion thereof, or sequences substantially corresponding to nucleotides 426 to 446 of the vanB gene, the complement thereof, or a portion thereof; and
b) detecting or determining the presence or amount of amplified nucleic acid.

5. The method of claim 3 wherein one vanA-specific oligonucleotide primer comprises sequences corresponding to nucleotides 851 to 868 of the vanA gene or a portion thereof.

6. The method of claim 3 wherein one vanA-specific oligonucleotide primer comprises sequences corresponding to the complement of nucleotides 898 to 919 of the vanA gene or a portion thereof.

7. The method of claim 3 wherein the presence or amount of amplified nucleic acid is detected or determined with an oligonucleotide probe comprising sequences corresponding to nucleotides 870 to 896 of the vanA gene, the complement thereof or a portion thereof.

8. The method of claim 1 wherein one vanA-specific oligonucleotide probe comprises sequences corresponding to nucleotides 870 to 896 of the vanA gene, the complement thereof or a portion thereof.

9. The method of claim 8 wherein the amplified vanB nucleic acid is obtained by amplifying a biological sample comprising nucleic acid with at least one vanA-specific oligonucleotide primer comprising sequences corresponding to nucleotides 851 to 868 of the vanA gene or a portion thereof, or sequences corresponding to the complement of nucleotides 898 to 917 of the vanA gene or a portion thereof.

10. The method of claim 4 wherein one vanB-specific oligonucleotide primer comprises sequences corresponding to nucleotides 387 to 404 of the vanB gene or a portion thereof.

11. The method of claim 4 wherein one vanB-specific oligonucleotide primer comprises sequences corresponding to the complement of nucleotides 426 to 446 of the vanB gene or a portion thereof.

12. The method of claim 4 wherein the presence or amount of amplified nucleic acid is detected or determined with an oligonucleotide probe comprising sequences corresponding to nucleotides 406 to 423 of the vanB gene, the complement thereof or a portion thereof.

13. The method of claim 2 wherein one vanB-specific oligonucleotide probe comprises sequences corresponding to nucleotides 406 to 423 of the vanB gene, the complement thereof or a portion thereof.

14. The method of claim 13 wherein the amplified vanB nucleic acid is obtained by amplifying a biological sample comprising nucleic acid with at least one vanB-specific oligonucleotide primer comprising sequences corresponding to nucleotides 387 to 404 of the vanB gene or a portion thereof, or sequences corresponding to the complement of nucleotides 426 to 446 of the vanB gene or a portion thereof.

15. The method of claim 1, 2, 3 or 4 wherein the sample is a physiological sample.

16. The method of claim 15 wherein the sample is a peri-rectal sample.

17. The method of claim 1, 7 or 8 further comprising contacting a corresponding sample with a probe which is not a vanA-specific probe.

18. The method of claim 1, 7 or 8 further comprising contacting the sample with a probe which is not a vanA-specific probe.

19. The method of claim 17 or 18 further comprising comparing the presence or amount of hybrid formation with the vanA-specific oligonucleotide probe to the presence or amount of hybrid formation between the sample contacted with the non-vanA probe.

20. The method of claim 2, 12, or 13 further comprising contacting a corresponding sample with a probe which is not a vanB-specific probe.

21. The method of claim 2, 12, or 13 further comprising contacting the sample with a probe which is not a vanB-specific probe.

22. The method of claim 20 or 21 further comprising comparing the presence or amount of hybrid formation with the vanB probe to the presence or amount of hybrid formation between the sample contacted with the non-vanB probe.

23. The method of claim 17 or 18 wherein the non-vanA probe is a vanB-specific probe.

24. The method of claim 20 or 21 wherein the non-vanB probe is a vanA-specific probe.

25. The method of claim 7, 8, 12 or 13 wherein the probe is labeled.

26. The method of claim 23 wherein the vanA-specific probe is labeled with a different label than the vanB-specific probe.

27. The method of claim 24 wherein the vanB-specific probe is labeled with a different label than the vanA-specific probe.

28. A method to detect vanA nucleic acid and vanB nucleic acid in a sample, comprising:
a) contacting a sample suspected of comprising amplified vanA nucleic acid or amplified vanB nucleic acid with at least one vanA-specific oligonucleotide probe and with at least one vanB-specific oligonucleotide probe under high stringency hybridization conditions effective to form a hybrid between the vanA-specific oligonucleotide probe and amplified vanA nucleic acid and between the vanB-specific oligonucleotide probe and amplified vanB nucleic acid, wherein the vanA-specific oligonucleotide probe comprises sequences which include sequences substantially corresponding to nucleotides 870 to 896 of the vanA gene, the complement thereof, or a portion thereof, sequences substantially corresponding to nucleotides 851 to 868 of the vanA gene, the complement thereof, or a portion thereof, or sequences substantially corresponding to nucleotides 898 to 917 of the vanA gene, the complement thereof, or a portion thereof, and wherein the vanB-specific oligonucleotide probe comprises sequences which include sequences substantially corresponding to nucleotides 387 to 404 of the vanB gene, the complement thereof, or a portion thereof, sequences substantially corresponding to nucleotides 406 to 423 of the vanB gene, the complement thereof, or a portion thereof, or sequences substantially corresponding to nucleotides 426 to 446 of the vanB gene, the complement thereof, or a portion thereof, and
b) detecting or determining the presence or amount of hybrid formation.

29. A method to detect vanA nucleic acid and vanB nucleic acid in a sample, comprising:
a) contacting a biological sample suspected of comprising vanA or vanB nucleic acid with at least one vanA-specific oligonucleotide primer under conditions effective to amplify vanA nucleic acid and with at least one vanB-specific oligonucleotide primer under conditions effective to amplify vanB nucleic acid, wherein the vanA-specific oligonucleotide primer comprises sequences which include sequences substantially corresponding to nucleotides 870 to 896 of the vanA gene, the complement thereof, or a portion thereof, sequences substantially corresponding to nucleotides 851 to 868 of the vanA gene, the complement thereof, or a portion thereof, or sequences substantially corresponding to nucleotides 898 to 917 of the vanA gene, the complement thereof, or a portion thereof, and wherein the vanB-specific oligonucleotides primer comprises sequences which include sequences substantially corresponding to nucleotides 387 to 404 of the vanB gene, the complement thereof, or a portion thereof, sequences substantially corresponding to nucleotides 406 to 423 of the vanB gene, the complement thereof, or a portion thereof, or sequences substantially corresponding to nucleotides 426 to 446 of the vanB gene, the complement thereof, or a portion thereof, and
b) detecting or determining the presence or amount of amplified nucleic acid.

30. The method of claim 29 wherein the presence or amount of amplified nucleic acid is detected with at least one vanA-specific oligonucleotide probe and at least one vanB-specific oligonucleotide probe.

31. The method of claim 28 or 30 wherein the at least one vanA-specific oligonucleotide probe and the at least one vanB-specific oligonucleotide probe have different labels.

32. An oligonucleotide composition comprising a first oligonucleotide comprising sequences substantially corresponding to nucleotides 870 to 896 of the vanA gene, the complement thereof, or a portion thereof, an oligonucleotide comprising sequences substantially corresponding to nucleotides 851 to 868 of the vanA gene the complement thereof, or a portion thereof, an oligonucleotide comprising sequences substantially corresponding to nucleotides 898 to 917 of the vanA gene, the complement thereof, or a portion thereof, or a combination thereof, wherein the oligonucleotide hybridizes under stringent hybridization conditions to vanA DNA.

33. An oligonucleotide composition comprising an oligonucleotide comprising sequences substantially corresponding to nucleotides 387 to 404 of the vanB gene, the complement thereof, or a portion thereof, an oligonucleotide comprising sequences substantially corresponding to nucleotides 406 to 423 of the vanB gene the complement thereof, or a portion thereof, an oligonucleotide comprising sequences substantially corresponding to nucleotides 426 to 446 of the vanB gene, the complement thereof, or a portion thereof, or a combination thereof, wherein the oligonucleotide hybridizes under stringent hybridization conditions to vanB DNA.

34. The oligonucleotide composition of claim 32 wherein at least one oligonucleotide has the length and sequence of any of SEQ ID NOs:2-4.

35. The oligonucleotide composition of claim 33 wherein at least one oligonucleotide has the length and sequence of any of SEQ ID NOs:6-9.

36. The oligonucleotide composition of claim 32 or 33 wherein the oligonucleotide is labeled.

37. A kit comprising an oligonucleotide specific for a vanA gene and/or a vanB gene in a test sample, comprising an oligonucleotide comprising sequences substantially corresponding to nucleotides 870 to 896 of the vanA gene, the complement thereof, or a portion thereof, or an oligonucleotide comprising sequences substantially corresponding to nucleotides 406 to 423 of the vanB gene, the complement thereof, or a portion thereof, wherein the oligonucleotide hybridizes under stringent hybridization conditions to vanA DNA or vanB DNA.

38. The kit of claim 37 further comprising at least one non-vanA or one non-vanB probe.

39. The kit of claim 37 further comprising an oligonucleotide comprising sequences substantially corresponding to nucleotides 387 to 404 of the vanB gene, the complement thereof, or a portion thereof, or an oligonucleotide comprising sequences substantially corresponding to nucleotides 426 to 446 of the vanB gene, the complement thereof, or a portion thereof.

40. The kit of claim 37 further comprising an oligonucleotide comprising sequences substantially corresponding to nucleotides 851 to 868 of the vanA gene, the complement thereof, or a portion thereof, or an oligonucleotide comprising sequences substantially corresponding to nucleotides 868 to 917 of the vanA gene, the complement thereof, or a portion thereof, or a combination thereof.

41. The kit of claim 37 wherein at least one oligonucleotide is labeled.

42. A kit comprising one or more oligonucleotides specific for a vanA gene in a test sample, comprising: an oligonucleotide comprising sequences substantially corresponding to nucleotides 851 to 868 of the vanA gene, the complement thereof, or a portion thereof, or an oligonucleotide comprising sequences substantially corresponding to nucleotides 898 to 917 of the vanA gene, the complement thereof, or a portion thereof, or a combination thereof.

43. A kit comprising one or more oligonucleotides specific for a vanB gene in a test sample, comprising: an oligonucleotide comprising sequences substantially corresponding to nucleotides 645 to 645 of the vanB gene, the complement thereof, or a portion thereof, or an oligonucleotide comprising sequences substantially corresponding to nucleotides 426 to 446 of the vanB gene, the complement thereof, or a portion thereof, or a combination thereof.
